(19) United States
(12) Patent Application Publication
(10) Pub. No.: US 2021/0213056 A1
(43) Pub. Date: Jul. 15, 2021

(54) MESENCHYMAL STROMAL CELL EXOSOME-TREATED MONOCYTES AND USES THEREOF

(71) Applicant: Children's Medical Center Corporation, Boston, MA (US)

(72) Inventors: Stella Kourembanas, Newton, MA (US); S. Alexander Mitsialis, Newton, MA (US)

(73) Assignee: Children's Medical Center Corporation, Boston, MA (US)

(21) Appl. No.: 17/053,752

(22) PCT Filed: May 9, 2019
(86) PCT No.: PCT/US19/31467

§ 371 Date: Nov. 6, 2020

Related U.S. Application Data
(60) Provisional application No. 62/669,324, filed on May 9, 2018.

(51) Int. Cl. A61K 35/15; A61P 37/02; A61K 45/06; C12N 5/0786

(52) U.S. Cl. CPC A61K 35/15; C12N 5/0645; A61K 45/06; A61P 37/02

(57) ABSTRACT

Provided herein are methods of modulating monocyte phenotypes using isolated mesenchymal stem cell (MSC) exosomes. Monocytes treated with MSC exosomes can be used to treat fibrotic disease and autoimmune diseases.

### RELATED APPLICATION

This application claims the benefit under 35 U.S.C. § 119(e) to U.S. Provisional Application No. 62/669324, filed May 9, 2018, and entitled “MESENCHYMAL STROMAL CELL EXOSOME-TREATED MONOCYTES AND USES THEREOF,” the entire contents of which are incorporated herein by reference.

### BACKGROUND

Idiopathic pulmonary fibrosis (IPF) is a chronic progressive respiratory disease with a prevalence of 0.5 to 27.9 per 100,000 person years. The lack of complete understanding of the underlying mechanism of this disease, may have contributed to the paucity of successful therapies. Despite two newly approved drugs, IPF remains fatal with a five-year survival rate of less than 10%.

### SUMMARY

It was shown herein that, a single intravenous (IV) dose of mesenchymal stem cell (MSC) exosomes reverts bleomycin-induced pulmonary fibrosis, at least partly through the modulation of monocyte phenotypes in the bone marrow and reduction of alveolar epithelial cell (AEC) apoptosis. Further, monocytes treated with MSC exosomes, when administered to a subject having pulmonary fibrosis, were therapeutically effective against the disease.

Accordingly, provided herein, in some aspects, are methods of regulating a monocyte phenotype, the method comprising contacting a monocyte with an isolated mesenchymal stem cell (MSC) exosome. In some embodiments, the monocyte is from bone marrow.

In some embodiments, the isolated MSC exosome is isolated from MSC-conditioned media. In some embodiments, the MSC is from Wharton's Jelly, bone marrow, or adipose tissue. In some embodiments, the isolated MSC exosome is substantially free of protein contaminants. In some embodiments, the isolated MSC exosome has a diameter of about 50-150 nm.

In some embodiments, the contacting is in vitro. In some embodiments, the contacting is ex vivo. In some embodiments, the contacting is in vivo. In some embodiments, the contacting is for at least 2 hours.

In some embodiments, the monocyte is pro-inflammatory prior to being contacted with the isolated MSC exosome, and is regulatory after being contacted with the isolated MSC exosome.

Other aspects of the present disclosure provide methods of treating a fibrotic disease or an autoimmune disease, the method comprising administering to a subject in need thereof an effective amount of a monocyte, wherein the monocyte is treated with an isolated mesenchymal stem cell (MSC) exosome prior to being administered.

In some embodiments, the method further comprises isolating the monocyte prior to treating the monocyte with the MSC exosome. In some embodiments, the monocyte is isolated from the subject. In some embodiments, the monocyte is isolated from the bone marrow of the subject.

In some embodiments, the monocyte is treated with the MSC exosome for at least 2 hours prior to being administered to the subject. In some embodiments, the monocyte is administered systemically. In some embodiments, the monocyte is administered via intravenous infusion. In some embodiments, the monocyte is administered intratracheally or intranasally. In some embodiments, the monocyte is administered once to the subject. In some embodiments, the monocyte is administered multiple times to the subject.

In some embodiments, the method further comprises administering to the subject an effective amount of a second agent. In some embodiments, the second agent is an isolated MCS exosome. In some embodiments, the second agent is nintedanib, Pirfenidone, an anti-fibrotic agent, an immunosuppressant, and/or an anti-inflammatory agent.

In some embodiments, the fibrotic disease is selected from the group consisting of: systemic sclerosis; liver fibrosis, heart fibrosis, kidney fibrosis, and myelofibrosis. In some embodiments, the fibrotic disease is pulmonary fibrosis. In some embodiments, the pulmonary fibrosis is idiopathic pulmonary fibrosis (IPF). In some embodiments, the monocyte reduces inflammation associated with the fibrotic disease. In some embodiments, the monocyte reduces apoptosis associated with the fibrotic disease.

In some embodiments, the subject is a mammal. In some embodiments, the subject is a human subject. In some embodiments, the human is a neonate, an infant, or an adult. In some embodiments, the human subject is less than four weeks of age. In some embodiments, the human subject is four weeks to 3 years of age. In some embodiments, the human subject is 3-18 years of age. In some embodiments, the human subject is an adult.

In some embodiments, the human subject is born prematurely. In some embodiments, the human subject was born before 37 weeks of gestation. In some embodiments, the human subject was born before 26 weeks of gestation.

In some embodiments, the subject is a rodent. In some embodiments, the rodent is a mouse or a rat.

In some embodiments, the monocyte is pro-inflammatory prior to being treated with the isolated MSC exosome, and is regulatory after being treated with the isolated MSC exosome.

Other aspects of the present disclosure provide monocytes treated with an isolated mesenchymal stem cell (MSC) exosome. In some embodiments, the monocyte is from bone marrow. In some embodiments, the isolated MSC exosome is isolated from MSC-conditioned media. In some embodiments, the MSC is from Wharton's Jelly, bone marrow, or adipose tissue. In some embodiments, the monocyte is pro-inflammatory prior to being treated with the isolated MSC exosome, and is regulatory after being treated with the isolated MSC exosome.

Compositions comprising the monocytes described herein are also provided. In some embodiments, the composition further comprises a second agent. In some embodiments, the composition is a pharmaceutical composition. In some embodiments, the composition further comprises a pharmaceutically acceptable carrier.

Further provided herein are uses of the monocyte or the composition comprising the monocytes described herein for treating a fibrotic disease or an autoimmune disease.

The monocyte or the composition comprising the monocytes described herein may also be used use in the manufacturing of a medicament for treating a fibrotic disease or an autoimmune disease.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. The patent or application file contains at least one drawing executed in color. Copies of this patent or patent application publication with color drawing(s) will be provided by the Office upon request and payment of the necessary fee. In the drawings:

FIGS. 1A to 1D show that MEx treatment at the beginning of inflammation prevents fibrosis. (FIG. 1A) Ten to fourteen-week old C57BL/6 mice received endotracheal bleomycin (60 μg) or 0.9% normal saline (NS) on day 0 followed by a bolus dose of IV MEx (Bleo+MEx), NS (bleo+NS), FEx (Bleo+FEx), or iodixanol (IDX 1:9 dilution, bleo+IDX). Results were compared to control group who received either NS (vehicle, control) or NS followed by a dose of MEx (control+MEx). Mice were sacrificed on day 14. (FIG. 1B) Lung sections were stained with Masson's trichrome. Inserts were taken at 100× magnification. Bleo+NS, Bleo+FEx, Bleo+IDX showed architectural destruction, alveolar septal thickening and fibrotic changes. (FIG. 1C) Administration of MEx to bleomycin-treated mice substantially reduced fibrosis and alveolar distortion. Findings were similar to control or Control+Mex group. Lung fibrosis was measured at day 14 by Ashcroft score. (FIG. 1D) Collagen deposition was assessed by Sircol assay and represented as mg/ml of left lung homogenate. n=3-4 per group, *p<0.05; ****p<0.0001 vs. bleomycin-treated group. Scale bar=100 μm.

FIGS. 2A to 2E show that MEx modulates alveolar macrophage phenotypes and blunt inflammation. Whole lung RT-qPCR shows an increase in the expression of macrophage Ccl-2 and Arginase-1 (Arg1) markers at day 7 (FIG. 2A) and day 14 (FIG. 2B), while their level was similar to control with MEx treatment. Interleukin-6 expression showed similar trend but its reduction with MEx treatment did not reach statistical significance. Levels of TGF-remained unchanged between the three groups. Results are expressed relative to control expression. Mean±SEM, n=4-8 per group. *p<0.05; **p<0.01 vs. bleomycin-exposed mice. (FIGS. 2C and 2D) Immunofluorescence (IF) analysis of lung sections using antibodies against markers of M2-like activation Arg1 (green) and CD206 (red) shows an increase in mean fluorescent intensity (MFI) in bleomycin mice, while the intensity was similar to control levels with MEx treatment. Nuclei staining performed with Dapi. Images obtained at x10 magnification. Mean fluorescent intensity normalized for cell number (Dapi stain). Analysis performed was by image J software. N=5 per group, *p<0.05; **p<0.01 vs. bleomycin-exposed mice. (FIG. 2E) Cumulative data and representative graph depicts the percentage of CD206^{+ve} alveolar macrophages (AM) (CD45^{+ve}CD11b^{-ve}CD11c^{+ve}CD206^{+ve} cells). Number of CD206^{+ve} AMs reduced with MEx treatment but did not reach statistical significance compared to the bleomycin-exposed group. Representative histogram normalized to mode. Mean±SEM of n=4-5 per group, **p<0.01 vs. bleomycin-exposed mice. Abbreviations: Dapi, 40,6-diamidino-2-phenylindole.

FIGS. 3A to 3F show that MEx modulates monocyte and macrophage phenotype at a systemic level MEx restore alveolar macrophage and inflammatory monocyte populations in the lung. (FIG. 3A) Cytometric analysis in whole lungs 7 days after injury showed a decrease in the AM number (represented as CD45^{+ve}CD11b^{-ve}CD11c^{+ve} cells). (FIG. 3B) This was associated with an increase in Ly6Chi infiltrating or classical monocytes (Ly6ChiCCR-2^{+ve}). (FIG. 3C) On day 14 AM number increased and (FIG. 3D) classical monocytes number decrease to approximately half of the level observed in NS-treated (control) group of mice (Mean difference: 1.7%±0.44, p<0.01). MEx therapy not only led to the restoration of the AM population number, but also modulated the monocyte phenotypes in the lung to levels comparable to control group analyzed at day 7 and 14. Mean±SEM of n=4-5 per group, *p<0.05; **p<0.01; ***p<0.001 vs. bleomycin-exposed mice. Gating strategy was performed according to fluorescence minus one controls (See FIG. 8). To investigate the systemic effects of MEx, the myeloid signature of the bone marrow was analyzed by flow cytometry. Despite similar numbers of CD45⁴ cells in the three groups (data not shown), (FIGS. 3E and 3F) classical monocytes increased in bleomycin-exposed group of mice (Mean difference: 17.6%±3.6, p<0.001 vs. bleomycin-exposed mice), but regulatory monocytes exhibited a 2-fold decrease (Mean difference: 18%±5.7, p<0.05 vs. bleomycin-exposed mice) in bleomycin-exposed mice compared to control mice. Whereas, MEx therapy led to a decrease in inflammatory monocytes and a shift from inflammatory to regulatory (Ly6ClowCCR-2-ve) phenotype, similar to levels observed in control mice (Mean difference: 10.25%±4.2, p<0.05 and 13.39%±5.76, p<0.05 vs. bleomycin-exposed mice). n=4-7 per group, *p<0.05; **p<0.01; ***p<0.001 vs. bleomycin-exposed mice.

FIGS. 4A to 4F show that adoptive transfer of MEx-pretreated bone marrow derived monocytes protects mice from pulmonary fibrosis. The potential therapeutic effects of ex vivo treated BMDMo and AMs in the prevention of fibrosis was explored. (FIG. 4A) BMDMo were isolated from 6-8-wks-old FVB mice, cultured ex vivo for 3 days and treated with MEx (equivalent to EVs produced by 1×10⁶ MSCs per 100 mm plate) or media alone on day 1, D1 and day 2, D2 and stained with Dil on day 3, D3. Cells were adoptively transferred intravenously at a one-to-one ratio on days 0 and 3 to C57BL/6 mice following endotracheal instillation of bleomycin. Mice were sacrificed at day 14. Data was compared to bleomycin-exposed mice who had received NS only (Bleomycin) (FIG. 4B) Flow cytometric analysis of BMDMo after 3 days of culture showed more than 90% CD45+veCD11b+ve cells. (FIG. 4C) Dil-labeled BMDMo were detected in the lung 14 days after injection. Images obtained at ×20 magnification. (FIGS. 4D to 4F) Fibrosis was ameliorated in mice that received MEx-pretreated monocytes (BMDMo+MEx) compared to NS (Bleomycin). Mice who were injected with MEx-treated AM (AM+MEx) exhibited substantial fibrotic changes. The administration of untreated-BMDMo (BMDM+Media) led to mild amelioration of fibrosis and collagen levels compared to NS-treated group of mice. The reduction in collagen deposition did not reach statistical significance compared to NS-treated mice. Similar results were noted at collagen level. Arrow marks the Dil-labeled monocytes. Between group comparison: *p<0.05, **p<0.01, ***p<0.001, ****p<0.001. Scale bar=100 μm. Abbreviations: Dil, 1,1′-Dioctadecyl-3,3,3′,3′-Tetramethylindocarbocyanine Perchlorate (‘Dil’; DilC18(3))

FIGS. 5A to 5D shows that MEx therapy decreases apoptosis. (FIGS. 5A and 5B) Tunel staining in whole lung sections shows increase in apoptosis (green) in the bleomycin-exposed group of mice compared to control (NS) and bleomycin+MEx. Nuclei were stained with Dapi. Images obtained at ×20 magnification. MFI quantified using image J software and normalized for Dapi. *p<0.05, **p<0.01 vs bleomycin-exposed mice (FIG. 5C) Annexin V/PI staining in whole lungs shows an increase in apoptosis (Annexin V+PI−) in bleomycin-exposed mice compared to control and bleomycin+MEx mice. (FIG. 5D) In vitro apoptosis was measured using Caspase-Glo® 3/7 Assay. More apoptosis is noted in Bleomycin-exposed human alveolar epithelial cells. This effect is abrogated with MEx therapy. Relative luminescence unit was used as a representative of apoptosis, Y axis represents luminescence relative to control. n=8 per group, **p<0.01; ****P<0.0001 vs bleomycin-exposed mice.

FIGS. 6A to 6C show the purification, isolation and characterization of exosomes. Conditioned media (CM) from BMSCs or HDFs was differentially centrifuged and concentrated through tangential flow filtration. Concentrated (50×) CM was floated on an iodixanol (OptiPrep™ IDX) cushion gradient. Purified EV population in fraction 9 was used for analysis. (FIG. 6A) Heterogeneous EV morphology seen on transmission electron microscopy (TEM) (×30,000 g, scale bar=100 nm). (FIG. 6B) Nanoparticle tracking analysis (NTA) was used to assess EV concentration. Representative size distribution of BMSC-EVs and HDF-EVs in fraction 9 gradient. (FIG. 6C) Western blot analysis of IDX cushion gradient fractions (7-10), using antibodies to exosomal markers flotillin (FLOT-1), CD63 & Alix.

FIGS. 7A to 7D show that MEx treatment at the end of inflammation reverts fibrosis. (FIG. 7A) MEx were administered 7 days after the administration of bleomycin and mice were sacrificed on day 14. (FIGS. 7B and 7C) Lung sections from Control, Bleomycin and Bleo+MEx mice were analyzed for histology and (FIG. 7D) collagen deposition. MEx therapy led to reduction in fibrosis and collagen deposition on day 7. Data represent mean±SEM of n=4 per group, *p<0.05; ****p<0.0001 vs. bleomycin-exposed mice. Scale bar=100 μm.

FIG. 8 shows the representative in vivo gating strategy of lung macrophage, monocyte and bone marrow derived monocytes. Cells were isolated from whole lung after enzymatic digestion. Lung aggregates and cell debris were excluded based on forward and side scatter parameters. Immune cells were identified by CD45 staining. Alveolar macrophages (AM) were identified using a sequential gating strategy to identify CD45^{+ve}CD11b^{-ve}CD11c^{+ve} population. Subsequent gating was performed on CD206^{+ve} AMs. In order to identify monocyte subpopulation, sequential gating strategy was performed on non-alveolar macrophage subset of CD45^{+ve} cells (CD11b^{int} CD11C^{low}) and further gated for CCR-2^{+ve}L y6C^{high} and CCR-2^{-ve}Ly6C^{low} population to reflect classical or non-classical monocyte phenotype respectively. BMDMo gating strategy was similar to above, with the exclusion of CD11c and CD206 (markers of AMs) staining. Gating strategy performed according to Fluorescence-minus-one controls.

FIG. 9 shows that labeled-MEx can be detected in the bone marrow. Membrane dye-labeled EVs were IV injected into mice, and the animals were sacrificed 2 hours after injection. MEx were detected in the BM cytospins (Labeled-MEx). Injected free dye and dye-stained EV free supernatant were used as controls. Counterstaining performed with Dapi. Images were obtained at ×60 magnification.

### DETAILED DESCRIPTION OF CERTAIN EMBODIMENTS

The present disclosure is based, at least in part, on the finding that mesenchymal stromal cell (also termed herein interchangeably as “mesenchymal stem cell” or “MSC”) exosomes (also termed “Mex” herein), when administered to a subject (e.g., systemically), can modulate monocyte phenotypes in the bone marrow, resulting in a larger subpopulation of regulatory monocytes instead of pro-inflammatory monocytes. Further, monocytes (e.g., bone marrow-derived monocytes) treated with MSC exosomes in vitro, when administered to subjects having pulmonary fibrosis, have therapeutic effects on fibrotic lungs.

Some aspects of the present disclosure provide monocytes treated with isolated mesenchymal stem cell (MSC) exosomes. A “monocyte” is a type of leukocyte (also called “white blood cell”) that can differentiate into macrophages and myeloid lineage dendritic cells. In vertebrates, monocytes are part of the innate immune system but can also influence the process of adaptive immunity.

Monocytes compose 2% to 10% of all leukocytes in the human body and serve multiple roles in immune function, e.g., without limitation, replenishing resident macrophages under normal conditions; migration in response to inflammation signals from sites of infection in the tissues; and differentiation into macrophages or dendritic cells to effect an immune response.

Monocytes are heterogeneous populations of cells, and can be divided into subpopulations with different phenotypes and functions. In some embodiments, human monocytes are subdivided into phenotypically and functionally distinct subpopulations based on the expression of the lipopolysaccharide (LPS) receptor (CD14) and the CD16 (Fcgamma receptor III) (e.g., as described in Ziegler-Heitbrock et al., Blood, vol. 116, no. 16, pp. e74-e80, 2010 and Gordon et al., Nature Reviews Immunology, vol. 5, no. 12, pp. 953-964, 2005, incorporated herein by reference). In healthy individuals, approximately 80-90% of monocytes are highly CD14 positive and CD16 negative (CD14⁺⁺CD16⁻). The CD14⁺⁺CD16⁻ monocytes are termed “classical monocytes” or “regulatory monocytes” herein. The remaining 10-20% of monocytes are CD16 positive and are classified as “proinflammatory monocytes.” Proinflammatory monocytes can further subdivided into CD14⁺⁺CD16⁺ and CD14⁺CD16⁺⁺ cells, which are The CD14⁺⁺CD16⁺ monocytes are also termed “intermediate monocytes;” and the CD14⁺CD16⁺⁺ monocytes are also termed “nonclassical monocytes.” Compared with CD16 negative conventional monocytes, CD16 positive monocytes (proinflammatory monocytes), express higher levels of major histocompatibility complex (MHC) class II antigens, adhesion molecules, chemokine receptors, and proinflammatory cytokines such as TNF-α, but lower levels of the anti-inflammatory cytokine (e.g., IL-10) (e.g., as described in Kawanaka et al., Arthritis & Rheumatism, vol. 46, no. 10, pp. 2578-2586, 2002 and Ziegler-Heitbrock et al., Immunology Today, vol. 17, no. 9, pp. 424-428, 1996, incorporated herein by reference). Proinflammatory monocytes are elevated in various pathologic conditions, including inflammatory and infectious diseases, cancer, and in coronary heart disease. In mice, monocytes can also be divided in two subpopulations: proinflammatory monocytes (Cx3CR1^{low}, CCR2⁺, Ly6C^{high}), which are equivalent to human proinflammatory monocytes; and regulatory monocytes (Cx3CR1^{high}, CCR2⁻, Ly6C^{low}), which are equivalent to human CD14⁺⁺CD16⁻ monocytes.

Monocytes are produced by the bone marrow from precursors called monoblasts, bipotent cells that differentiated from hematopoietic stem cells. Monocytes circulate in the bloodstream for about one to three days and then typically move into tissues throughout the body where they differentiate into macrophages and dendritic cells. In some embodiments, the monocytes treated with MSC exosomes described herein are from bone marrow (e.g., isolated from bone marrow). In some embodiments, the monocytes treated with MSC exosomes described herein are from a specific tissue (e.g., isolated from a specific tissue such as lungs).

An “exosome” is a membrane (e.g., lipid bilayer) vesicle that is released from a cell (e.g., any eukaryotic cell). Exosomes are present in eukaryotic fluids, including blood, urine, and cultured medium of cell cultures. The exosomes of the present disclosure are released from mesenchymal stem cells (MSCs) and are interchangeably termed “mesenchymal stem cell exosomes” or “MSC exosomes.”

A “mesenchymal stem cell (MSC)” is a progenitor cell having the capacity to differentiate into neuronal cells, adipocytes, chondrocytes, osteoblasts, myocytes, cardiac tissue, and other endothelial or epithelial cells. (See for example Wang et al., Stem Cells 2004; 22(7); 1330-7; McElreavey; 1991 Biochem Soc Trans (1); 29s; Takechi, Placenta 1993 March/April; 14 (2); 235-45; Takechi, 1993; Kobayashi; Early Human Development; 1998; July 10; 51 (3); 223-33; Yen; Stem Cells; 2005; 23 (1) 3-9.) These cells may be defined phenotypically by gene or protein expression. These cells have been characterized to express (and thus be positive for) one or more of CD13, CD29, CD44, CD49a, b, c, e, f, CD51, CD54, CD58, CD71, CD73, CD90, CD102, CD105, CD106, CDw119, CD120a, CD120b, CD123, CD124, CD126, CD127, CD140a, CD166, P75, TGF-bIR, TGF-bIIR, HLA-A, B, C, SSEA-3, SSEA-4, D7 and PD-L1. These cells have also been characterized as not expressing (and thus being negative for) CD3, CD5, CD6, CD9, CD10, CD11a, CD14, CD15, CD18, CD21, CD25, CD31, CD34, CD36, CD38, CD45, CD49d, CD50, CD62E, L, S, CD80, CD86, CD95, CD117, CD133, SSEA-1, and ABO. Thus, MSCs may be characterized phenotypically and/or functionally according to their differentiation potential.

MSCs may be harvested from a number of sources including but not limited to bone marrow, adipose tissue, blood, periosteum, dermis, umbilical cord blood and/or matrix (e.g., Wharton's Jelly), and placenta. For example, MSCs can be isolated from commercially available bone marrow aspirates. Enrichment of MSCs within a population of cells can be achieved using methods known in the art including but not limited to fluorescence-activated cell sorting (FACS). Methods for harvesting MSCs are described in the art, e.g., in U.S. Pat. No. 5,486,359, incorporated herein by reference.

Commercially available media may be used for the growth, culture and maintenance of MSCs. Such media include but are not limited to Dulbecco's modified Eagle's medium (DMEM). Components in such media that are useful for the growth, culture and maintenance of MSCs, fibroblasts, and macrophages include but are not limited to amino acids, vitamins, a carbon source (natural and non-natural), salts, sugars, plant derived hydrolysates, sodium pyruvate, surfactants, ammonia, lipids, hormones or growth factors, buffers, non-natural amino acids, sugar precursors, indicators, nucleosides and/or nucleotides, butyrate or organics, DMSO, animal derived products, gene inducers, non-natural sugars, regulators of intracellular pH, betaine or osmoprotectant, trace elements, minerals, non-natural vitamins. Additional components that can be used to supplement a commercially available tissue culture medium include, for example, animal serum (e.g., fetal bovine serum (FBS), fetal calf serum (FCS), horse serum (HS)), antibiotics (e.g., including but not limited to, penicillin, streptomycin, neomycin sulfate, amphotericin B, blasticidin, chloramphenicol, amoxicillin, bacitracin, bleomycin, cephalosporin, chlortetracycline, zeocin, and puromycin), and glutamine (e.g., L-glutamine). Mesenchymal stem cell survival and growth also depends on the maintenance of an appropriate aerobic environment, pH, and temperature. MSCs can be maintained using methods known in the art, e.g., as described in Pittenger et al., Science, 284:143-147 (1999), incorporated herein by reference.

In some embodiments, the MSC exosomes used to treat the monocytes are isolated. As used herein, an “isolated exosome” is an exosome that is physically separated from its natural environment. An isolated exosome may be physically separated, in whole or in part, from tissue or cells with which it naturally exists (e.g., MSCs). In some embodiments, the isolated MSC exosomes are isolated from the culturing media of MSCs from human bone marrow, umbilical cord Wharton's Jelly, or adipose tissue. Such culturing media is termed “MSC-conditioned media” herein. In some embodiments, isolated exosomes may be free of cells such as MSCs, or it may be free or substantially free of conditioned media, or it may be free of any biological contaminants such as proteins. Typically, the isolated exosomes are provided at a higher concentration than exosomes present in un-manipulated conditioned media.

In some embodiments, the isolated MSC exosome described herein comprises one or more (e.g., 1, 2, 3, 4, 5, or more) known exosome markers. In some embodiments, the known exosome markers are selected from the group consisting of: FLOT1 (Flotillin-1, Uniprot ID: 075955), CD9 (CD9 antigen, Uniprot ID: P21926), and CD63 (CD63 antigen, Uniprot ID: P08962).

In some embodiments, the isolated MSC exosome is substantially free of contaminants (e.g., protein contaminants). The isolated MSC exosome is “substantially free of contaminants” when the preparation of the isolated MSC exosome contains fewer than 20%, 15%, 10%, 5%, 2%, 1%, or less than 1%, of any other substances (e.g., proteins). In some embodiments, the isolated MSC is “substantially free of contaminants” when the preparation of the isolated MSC exosome is at least 80%, at least 85%, at least 90%, at least 95%, at least 98%, at least 99%, at least 99.9% pure, with respect to contaminants (e.g., proteins).

“Protein contaminants” refer to proteins that are not associated with the isolated exosome and do not contribute to the biological activity of the exosome. The protein contaminants are also referred to herein as “non-exosomal protein contaminants.”

In some embodiments, the isolated MSC exosome used in accordance with the present disclosure has a diameter of about 30-150 nm. For example, the isolated MSC exosome may have a diameter of 30-150 nm, 30-140 nm, 30-130 nm, 30-120 nm, 30-110 nm, 30-100 nm, 30-90 nm, 30-80 nm, 30-70 nm, 30-60 nm, 30-50 nm, 30-40 nm, 40-150 nm, 40-140 nm, 40-130 nm, 40-120 nm, 40-110 nm, 40-100 nm, 40-90 nm, 40-80 nm, 40-70 nm, 40-60 nm, 40-50 nm, 50-150 nm, 50-140 nm, 50-130 nm, 50-120 nm, 50-110 nm, 50-100 nm, 50-90 nm, 50-80 nm, 50-70 nm, 50-60 nm, 60-150 nm, 60-140 nm, 60-130 nm, 60-120 nm, 60-110 nm, 60-100 nm, 60-90 nm, 60-80 nm, 60-70 nm, 70-150 nm, 70-140 nm, 70-130 nm, 70-120 nm, 70-110 nm, 70-100 nm, 70-90 nm, 70-80 nm, 80-150 nm, 80-140 nm, 80-130 nm, 80-120 nm, 80-110 nm, 80-100 nm, 80-90 nm, 90-150 nm, 90-140 nm, 90-130 nm, 90-120 nm, 90-110 nm, 90-100 nm, 100-150 nm, 100-140 nm, 100-130 nm, 100-120 nm, 100-110 nm, 110-150 nm, 110-140 nm, 110-130 nm, 110-120 nm, 120-150 nm, 120-140 nm, 120-130 nm, 130-150 nm, 130-140 nm, or 140-150 nm. In some embodiments, the isolated MSC exosome may have a diameter of about 50 nm, 60 nm, 70 nm, 80 nm, 90 nm, 100 nm, 110 nm, 120 nm, 130 nm, 140 nm, or 150 nm. In some embodiments, the isolated MSC exosomes exhibit a biconcave morphology.

As described herein, the isolated MSC exosomes can be used to treat the monocytes to modulate the monocyte phenotype (e.g., both in vitro and in vivo such as in the bone marrow). “Treat a monocyte with an isolated MSC exosome” means contacting the monocyte with a MSC exosome (e.g., for a period of time). In some embodiments, the treating (i.e., contacting) is carried out in vitro. For example, monocytes may be cultured in vitro and isolated MSC exosomes may be added to the culture such that the monocytes contact the isolated MSC exosomes. In some embodiments, the treating (i.e., contacting) is carried out ex vivo. For example, monocytes may be isolated from the bone marrow of a subject and isolated MSC exosomes may be added to the monocytes such that the monocytes contact the isolated MSC exosomes. In some embodiments, the treating (i.e., contacting) is carried out in vivo. For example, the isolated MSC exosomes may be administered to a subject (e.g., via intravenous injection), reach the one marrow, and contact the monocytes in the bone marrow.

In some embodiments, the monocyte is treated (i.e., contacted) with the MSC exosome for at least 1 hour (e.g., at least 1, at least 2, at least 3, at least 4, at least 5, at least 6, a least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100 hours, or longer). In some embodiments, the monocyte is treated (i.e., contacted) with the MSC exosome for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 hours, or longer.

In some embodiments, the monocyte has been polarized to a pro-inflammatory state as a result of environmentally or developmentally-precipitated injury, and its polarity is modulated to a regulatory phenotype upon contact with the isolated MSC exosome. In some embodiments, the monocyte is a pro-inflammatory monocyte prior to being treated (i.e., contacted) with the isolated MSC exosome, and is a regulatory monocyte after being treated (i.e., contacted) with the isolated MSC exosome. In some embodiments, a mixture of pro-inflammatory monocytes and regulatory monocytes are contacted with isolated MSC exosomes and the treating results in a higher ratio (e.g., at least 10% higher) of regulatory monocytes in the mixture, being treated with isolated MSC exosomes. For example, the ratio of regulatory monocytes may be at least 10%, at least 20%, at least 30%, at least 40%, at least 50%, at least 60%, at least 70%, at least 80%, at least 90%, at least 100%, at least 2-fold, at least 5-fold, at least 10-fold, at least 100-fold, or higher after being treated with MSC exosomes, compared to before being treated with isolated MSC exosomes. In some embodiments, the ratio of regulatory monocytes is 10%, 20%, 30%, 40%, 50%, 60%, 70%, 80%, 90%, 100%, 2-fold, 5-fold, 10-fold, 100-fold, or higher after being treated with MSC exosomes, compared to before being treated with isolated MSC exosomes.

Further provided herein are uses of the monocytes treated with isolated MSC exosomes for treating a disease (e.g., a fibrotic disease such as pulmonary fibrosis or an autoimmune disease). In some embodiments, the monocytes treated with isolated MSC exosomes are used in the manufacturing of a medicament for treating a disease (e.g., a fibrotic disease or an autoimmune disease). Compositions comprising monocytes treated with isolated MSC exosomes are also provided. In some embodiments, the monocytes treated with isolated MSC exosomes are formulated in a composition for the treatment of a disease (e.g., a fibrotic disease or an autoimmune disease).

In some embodiments, the composition comprising monocytes treated with isolated MSC exosomes further comprises a second agent. In some embodiments, the second agent is a therapeutic agent effective against the diseases being treated by the monocytes. For example, the second agent may be any agent that can be used in the prevention, treatment and/or management of a fibrotic disease or an autoimmune disease such as those described herein. In some embodiments, the second agent is an isolated MSC exosome.

In some embodiments, the second agent is an agent that is known to have therapeutic effects against fibrotic diseases. Exemplary second agents that may be used to treat fibrotic diseases include, without limitation: nintedanib (a tyrosine kinase inhibitor), pirfenidone, an anti-fibrotic agent, and/or an anti-inflammatory agent. In some embodiments, for pulmonary fibrosis, other types of therapies, e.g., oxygen supplement, may be used in conjunction with the therapeutic agents described herein.

In some embodiments, the second agent is an agent that is known to have therapeutic effects against autoimmune diseases. Such agents include, without limitation, non-steroidal anti-inflammatory drugs, glucocorticoids, metrotrexate, leflunomide, anti-TNF biologicals (e.g., antibodies such as infliximab, adalimumab, golinumab, or certolizumab pegol). Drugs for treating autoimmune diseases are known in the art, e.g., as described in Li et al., Front Pharmacol. 2017; 8: 460, incorporated herein by reference.

In some embodiments, the monocytes treated with isolated MSC exosomes and the second agent are formulated in the same composition. In some embodiments, the monocytes treated with isolated MSC exosomes and the second agent are formulated in separate compositions. In some embodiments, the monocytes treated with isolated MSC exosomes and the second agent are administered to the subject simultaneously. In some embodiments, the monocytes treated with isolated MSC exosomes and the second agent are administered separately. In some embodiments, the monocytes treated with isolated MSC exosomes are administered before the second agent. In some embodiments, the monocytes treated with isolated MSC exosomes are administered after the second agent.

In some embodiments, the composition comprising the monocytes treated with isolated MSC exosomes is a pharmaceutical composition. In some embodiments, the composition further comprises pharmaceutically acceptable concentrations of salt, buffering agents, preservatives, or compatible carriers.

A pharmaceutically acceptable carrier is a pharmaceutically acceptable material, composition or vehicle, such as a liquid or solid filler, diluent, excipient, solvent or encapsulating material, involved in carrying or transporting a prophylactically or therapeutically active agent. Each carrier must be “acceptable” in the sense of being compatible with the other ingredients of the formulation and not injurious to the subject. Some examples of materials which can serve as pharmaceutically acceptable carriers include sugars, such as lactose, glucose and sucrose; glycols, such as propylene glycol; polyols, such as glycerin, sorbitol, mannitol and polyethylene glycol; esters, such as ethyl oleate and ethyl laurate; buffering agents, such as magnesium hydroxide and aluminum hydroxide; pyrogen-free water; isotonic saline; Ringer's solution; ethyl alcohol; phosphate buffer solutions; and other non-toxic compatible substances employed in pharmaceutical formulations.

The compositions may take such forms as water-soluble suspensions, solutions or emulsions in oily or aqueous vehicles, and may contain formulatory agents such as suspending, stabilizing and/or dispersing agents. Suitable lipophilic solvents or vehicles include fatty oils such as sesame oil, or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or agents which increase solubility. Alternatively, the exosomes may be in lyophilized or other powder or solid form for constitution with a suitable vehicle, e.g., sterile pyrogen-free water, before use.

Other aspects of the present disclosure provide methods of treating a disease (e.g., a fibrotic disease or an autoimmune disease), the method comprising administering to a subject in need thereof an effective amount of a monocyte, wherein the monocyte is treated with an isolated mesenchymal stem cell (MSC) exosome (e.g., for at least 2 hours) prior to being administered using the methods described herein. In some embodiments, the method further comprises isolating the monocytes from the subject (e.g., from the bone marrow of the subject) such that the monocytes can be treated with isolated MSC exosomes prior to administration to the subject.

“Treat” or “treatment” of a disease (e.g., a fibrotic disease or an autoimmune disease) includes, but is not limited to, preventing, reducing, or halting the development of a fibrotic disease or an autoimmune disease, reducing or eliminating the symptoms of a fibrotic disease or an autoimmune disease, or preventing a fibrotic disease or an autoimmune disease.

An “effective amount” is the amount of an agent that achieves the desired outcome. The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

In some embodiments, the effective amount is a dosage of an agent that causes no toxicity to the subject. In some embodiments, the effective amount is a dosage of an agent that causes reduced toxicity to the subject. Methods for measuring toxicity are well known in the art (e.g., biopsy/histology of the liver, spleen, and/or kidney; alanine transferase, alkaline phosphatase and bilirubin assays for liver toxicity; and creatinine levels for kidney toxicity).

A subject shall mean a human or vertebrate animal or mammal including but not limited to a rodent, e.g., a rodent such as a rat or a mouse, dog, cat, horse, cow, pig, sheep, goat, turkey, chicken, and primate, e.g., monkey. In some embodiments, the subject is human. In some embodiments, the subject is a companion animal. “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. The methods of the present disclosure are useful for treating a subject in need thereof. The subjects may be those that have a disease described herein amenable to treatment using the monocytes described in this disclosure, or they may be those that are at risk of developing such a disease.

In some embodiments, the subject is a human subject. In some embodiments, the subject is a human infant. For example, the subject may be a neonate and particularly neonates born at low gestational age. As used herein, a human neonate refers to a human from the time of birth to about 4 weeks of age. As used herein, a human infant refers to a human from about the age of 4 weeks of age to about 3 years of age. As used herein, low gestational age refers to birth (or delivery) that occurs before a normal gestational term for a given species. In humans, a full gestational term is about 40 weeks and may range from 37 weeks to more than 40 weeks. Low gestational age, in humans, akin to a premature birth is defined as birth that occurs before 37 weeks of gestation. The disclosure therefore contemplates prevention and/or treatment of subjects born before 37 weeks of gestation, including those born at even shorter gestational terms (e.g., before 36, before 35, before 34, before 33, before 32, before 31, before 30, before 29, before 28, before 27, before 26, or before 25 weeks of gestation).

For infants or neonates, the present disclosure contemplates their treatment even beyond the neonate stage and into childhood and/or adulthood. For example, in some embodiments, the subject treated using the methods of the present disclosure is 3-18 years of age. In some embodiments, the subject treated using the methods of the present disclosure may be 3-18, 3-17, 3-16, 3-15, 3-14, 3-13, 3-12, 3-11, 3-10, 3-9, 3-8, 3-7, 3-6, 3-5, 3-4, 4-18, 4-17, 4-16, 4-15, 4-14, 4-13, 4-12, 4-11, 4-10, 4-9, 4-8, 4-7, 4-6, 4-5, 5-18, 5-17, 5-16, 5-15, 5-14, 5-13, 5-12, 5-11, 5-10, 5-9, 5-8, 5-7, 5-6, 6-18, 6-17, 6-16, 6-15, 6-14, 6-13, 6-12, 6-11, 6-10, 6-9, 6-8, 6-7, 7-18, 7-17, 7-16, 7-15, 7-14, 7-13, 7-12, 7-11, 7-10, 7-9, 7-8, 8-18, 8-17, 8-16, 8-15, 8-14, 8-13, 8-12, 8-11, 8-10, 8-9, 9-18, 9-17, 9-16, 9-15, 9-14, 9-13, 9-12, 9-11, 9-10, 10-18, 10-17, 10-16, 10-15, 10-14, 10-13, 10-12, 10-11, 11-18, 11-17, 11-16, 11-15, 11-14, 11-13, 11-12, 12-18, 12-17, 12-16, 12-15, 12-14, 12-13, 13-18, 13-17, 13-16, 13-15, 13-14, 14-18, 14-17, 14-16, 14-15, 15-18, 15-17, 15-16, 16-18, 16-17, or 17-18 years of age. In some embodiments, the subject is an adult, e.g., 18 or more than 18 years of age.

Certain subjects may have a genetic predisposition to certain forms of the diseases (or conditions) described herein (for example, autoimmune diseases or fibrotic disease), and those subjects may also be treated according to the disclosure.

With respect to neonates and particularly low gestation age neonates, the disclosure contemplates administration of the monocytes treated with isolated MSC exosomes or the composition comprising such within 1 year, 11 months, 10 months, 9 months, 8 months, 7 months, 6 months, 5 months, 4 months, 3 months, 2 months, 1 month, 4 weeks, 3 weeks, 2 weeks, 1 week, 6 days, 5 days, 4 days, 3 days, 2 days, 1 day, 12 hours, 6 hours, 3 hours, or 1 hour of birth. In some embodiments, the monocytes treated with isolated MSC exosomes or the composition comprising such are administered within 1 hour of birth (e.g., within 1 hour, within 55 minutes, within 50 minutes, within 45 minutes, within 40 minutes, within 35 minutes, within 30 minutes, within 25 minutes, within 20 minutes, within 15 minutes, within 10 minutes, within 5 minutes, or within 1 minute). In some embodiments, the monocytes treated with isolated MSC exosomes or the composition comprising such monocytes is administered to the subject immediately after birth.

The present disclosure further contemplates administration of the monocytes treated with isolated MSC exosomes or the composition comprising such even in the absence of symptoms indicative of a disease or disorder as described herein.

In some embodiments, the monocytes treated with isolated MSC exosomes or the composition comprising such monocytes are administered to a subject (e.g., a human subject) once. In some embodiments, repeated administration of the monocytes treated with isolated MSC exosomes or the composition comprising such monocytes, including two, three, four, five or more administrations of the monocytes treated with isolated MSC exosomes or the composition comprising such monocytes, is contemplated. In some instances, the monocytes treated with isolated MSC exosomes or the composition comprising such may be administered continuously. Repeated or continuous administration may occur over a period of several hours (e.g., 1-2, 1-3, 1-6, 1-12, 1-18, or 1-24 hours), several days (e.g., 1-2, 1-3, 1-4, 1-5, 1-6 days, or 1-7 days) or several weeks (e.g., 1-2 weeks, 1-3 weeks, or 1-4 weeks) depending on the severity of the condition being treated. If administration is repeated but not continuous, the time in between administrations may be hours (e.g., 4 hours, 6 hours, or 12 hours), days (e.g., 1 day, 2 days, 3 days, 4 days, 5 days, or 6 days), or weeks (e.g., 1 week, 2 weeks, 3 weeks, or 4 weeks). The time between administrations may be the same or they may differ.

In some embodiments, the monocytes treated with isolated MSC exosomes or the composition comprising such monocytes are administered at least once within 24 hours of birth and then at least once more within 1 week of birth. In some embodiments, the monocytes treated with isolated MSC exosomes or the composition comprising such monocytes are administered at least once within 1 hour of birth and then at least once more within 3-4 days of birth.

The monocytes treated with isolated MSC exosomes or the composition comprising such monocytes may be administered by any route that effects delivery to the fibrotic organ and/or the bone marrow. Systemic administration routes such as intravenous injection or continuous infusion are suitable. Other administration routes that are also suitable include oral administration, intranasal administration, intratracheal administration, inhalation, intravenous administration, etc. Those of ordinary skill in the art will know the customary routes of administration.

The monocytes treated with isolated MSC exosomes or the composition comprising such monocytes, may be formulated for parenteral administration by injection, including for example by bolus injection or continuous infusion. Formulations for injection may be presented in unit dosage form, e.g., in ampoules or in multi-dose containers, with or without an added preservative. The compositions may take such forms as water-soluble suspensions, solutions or emulsions in oily or aqueous vehicles, and may contain formulatory agents such as suspending, stabilizing and/or dispersing agents. Suitable lipophilic solvents or vehicles include fatty oils such as sesame oil, or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or agents which increase solubility. Alternatively, the exosomes may be in lyophilized or other powder or solid form for constitution with a suitable vehicle, e.g., sterile pyrogen-free water, before use.

In some embodiments, if the second agent is not formulated in the same composition as the monocytes treated with isolated MSC exosomes, the method described herein further comprises administering an effective amount of the second agent (e.g., agents for treating a fibrotic disease or an autoimmune disease). The second agent may also be administered by any suitable route including systemic administration (e.g., intravenous infusion or injection), oral administration, intranasal administration, intratracheal administration, inhalation, etc. Those of ordinary skill in the art will know the customary routes of administration for such second agents.

A “fibrotic disease” or “fibrosis” refers to a condition manifested by the formation of excess fibrous connective tissue in an organ or tissue in a reparative or reactive process. Non-limiting examples of fibrotic diseases include: systemic sclerosis (Scleroderma), pulmonary fibrosis (e.g., cystic fibrosis or idiopathic pulmonary fibrosis), liver fibrosis (cirrhosis or biliary atresia, heart fibrosis (e.g., atrial fibrosis, endomyocardial fibrosis, or old myocardial infarction), brain fibrosis (e.g., glial scar), kidney fibrosis, and myelofibrosis. Other types of fibrotic diseases include, without limitation: arterial stiffness, arthrofibrosis (knee, shoulder, other joints), crohn's disease (intestine), dupuytren's contracture (hands, fingers), keloid (skin), mediastinal fibrosis (soft tissue of the mediastinum), myelofibrosis (bone marrow), peyronie's disease (penis), nephrogenic systemic fibrosis (skin), progressive massive fibrosis (lungs); a complication of coal workers' pneumoconiosis, retroperitoneal fibrosis (soft tissue of the retroperitoneum), scleroderma/systemic sclerosis (skin, lungs), and some forms of adhesive capsulitis (shoulder).

In some embodiments, the fibrotic disease is pulmonary fibrosis. “Pulmonary fibrosis” refers to a condition where lung tissue becomes damaged and scarred, causing thickening and stiffing of the lung tissue and reduced lung function. Pulmonary fibrosis can have a variety of cause. Pulmonary fibrosis is typically seen in subjects with bronchopulmonary dysplasia (BPD).

In some embodiments, the pulmonary fibrosis is idiopathic pulmonary fibrosis (IPF). Idiopathic pulmonary fibrosis is characterized by scarring or thickening of the lungs without a known cause. It occurs most often in persons 50-70 years of age. Its symptoms include shortness of breath, regular cough (typically a dry cough), chest pain, and decreased activity level. For fibrotic diseases (e.g., pulmonary fibrosis), administration of the monocytes treated with isolated MSC exosomes at the beginning or late stage of inflammation associated with the fibrosis are shown herein to both be therapeutically effective against the diseases.

In some embodiments, the monocyte treated with isolated MSC exosomes reduces inflammation associated with the fibrotic disease. One skilled in the art is familiar with methods of assessing the degree of inflammation in a fibrotic organ (e.g., the lung). In some embodiments, inflammation may be assessed by measuring the levels of biomarkers of inflammation in the fibrotic organ or in the blood. In some embodiments, inflammations in the fibrotic organ (e.g., the lung) is reduced by at least 20%, in subjects that have been administered the monocytes treated with isolated MSC exosomes, compared to in subjects that have not been administered the monocytes treated with isolated MSC exosomes. For example, inflammation may be reduced by at least 20%, at least 30%, at least 40%, at least 50%, at least 60%, at least 70%, at least 80%, at least 90%, at least 95%, at least 99%, or 100%, in subjects that have been administered the monocytes treated with isolated MSC exosomes, compared to in subjects that have not been administered the monocytes treated with isolated MSC exosomes. In some embodiments, inflammation is reduced by 20%, 30%, 40%, 50%, 60%, 70%, 80%, 90%, 95%, 99%, or 100%, in subjects that have been administered the monocytes treated with isolated MSC exosomes, compared to in subjects that have not been administered the monocytes treated with isolated MSC exosomes.

In some embodiments, the monocytes treated with isolated MSC exosomes reduces apoptosis of epithelial cells in the fibrotic organ (e.g., alveolar epithelial cells in the lung). “Apoptosis” refers to the death of cells that occurs as a normal and controlled part of an organism's growth or development. In some embodiments, apoptosis of epithelial cells in the fibrotic organ (e.g., alveolar epithelial cells in the lung) is considered “reduced” when the number of alveolar epithelial cells undergoing apoptosis is reduced by at least 20%, in subjects that have been administered the monocytes treated with the isolated MSC exosomes, compared to in subjects that have not been administered the monocytes treated with the isolated MSC exosomes. For example, apoptosis of epithelial cells in the fibrotic organ (e.g., alveolar epithelial cells in the lung) may be considered “reduced” when the number of alveolar epithelial cells undergoing apoptosis is reduced by at least 20%, at least 30%, at least 40%, at least 50%, at least 60%, at least 70%, at least 80%, at least 90%, at least 95%, at least 99%, or 100%, in subjects that have been administered the monocytes treated with isolated MSC exosomes, compared to in subjects that have not been administered the monocytes treated with isolated MSC exosomes. In some embodiments, apoptosis of epithelial cells in the fibrotic organ (e.g., alveolar epithelial cells in the lung) is considered “reduced” when the number of alveolar epithelial cells undergoing apoptosis is reduced by 20%, 30%, 40%, 50%, 60%, 70%, 80%, 90%, 95%, 99%, or 100%, in subjects that have been administered the monocytes treated with isolated MSC exosomes, compared to in subjects that have not been administered the monocytes treated with the MSC exosomes.

In some embodiments, the monocytes treated with isolated MSC exosomes reduces pulmonary fibrosis. Pulmonary fibrosis is considered “reduced” when the degree of pulmonary fibrosis (e.g., as indicated by collagen deposition on lung tissues) is reduced by at least 20%, in subjects that have been administered the monocytes treated with the MSC exosomes, compared to in subjects that have not been administered the monocytes treated with the MSC exosomes. For example, pulmonary fibrosis may be considered reduced when the degree of pulmonary fibrosis (e.g., as indicated by collagen deposition on lung tissues) is reduced by at least 20%, at least 30%, at least 40%, at least 50%, at least 60%, at least 70%, at least 80%, at least 90%, at least 95%, at least 99%, or 100%, in subjects that have been administered the monocytes treated with the MSC exosomes, compared to in subjects that have not been administered the monocytes treated with the MSC exosomes. In some embodiments, pulmonary fibrosis is considered reduced when the degree of pulmonary fibrosis (e.g., as indicated by collagen deposition on lung tissues) is reduced by 20%, 30%, 40%, 50%, 60%, 70%, 80%, 90%, 95%, 99%, or 100%, in subjects that have been administered the monocytes treated with the MSC exosomes, compared to in subjects that have not been administered the monocytes treated with the MSC exosomes.

An “autoimmune disease” is a condition in which your immune system mistakenly attacks your body. Normally, the immune system can tell the difference between foreign cells and your own cells. In an autoimmune disease, the immune system mistakes part of your body (e.g., joint or skin) as foreign. It releases proteins called autoantibodies that attack healthy cells. Some autoimmune diseases target only one organ. Type 1 diabetes damages the pancreas. Other diseases, like lupus, affect the whole body. Non-limiting examples of autoimmune diseases include: Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, Wegener's granulomatosis (or Granulomatosis with Polyangiitis (GPA)).

In some embodiments, the autoimmune disease is selected from the group consisting of: rheumatoid arthritis (RA), systemic lupus erythematosus (SLE), Myasthenia Gravis (MG), Graves Disease, Idiopathic Thrombocytopenia Purpura (ITP), Guillain-Barre Syndrome, autoimmune myocarditis, Membrane Glomerulonephritis, Type I or Type II diabetes, juvenile onset diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, and immune responses associated with delayed hypersensitivity mediated by cytokines, T-lymphocytes typically found in tuberculosis, sarcoidosis, and polymyositis, polyarteritis, cutaneous vasculitis, pemphigus (e.g., pemphigus vulgaris, pemphigus foliaceus or paraneoplastic pemphigus), pemphigoid, Goodpasture's syndrome, Kawasaki's disease, systemic sclerosis, anti-phospholipid syndrome, and Sjogren's syndrome.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

Idiopathic pulmonary fibrosis (IPF) is a chronic progressive respiratory disease whose underlying mechanism is incompletely understood and which currently lacks effective treatments. Despite promising results with mesenchymal stromal cell (MSC) treatment in the prevention of lung fibrosis, limitations of cell therapies continue to render cell-free therapies highly desirable. In pre-clinical models other than IPF, MSC-extracellular vesicles (EVs) or more specifically exosomes (MEx) isolated from MSC secretome, have been shown to act as the therapeutic vector. The effect of MEx, and their mechanism of action (MOA) in IPF are unknown.

Objectives: The efficacy and MOA of MEx in a bleomycin-IPF model was investigated. Methods: Exosomes isolated from human bone marrow MSCs (MEx) were injected into adult C57BL/6 mice 0 or 7 days following instillation of endotracheal bleomycin. Lungs and bone marrow-derived monocytes (BMDMo) were harvested on day 7 and 14 for histologic, gene expression or cytometric analysis.

### Measurements and Main Results

MEx treatment concurrent with or 7 days after bleomycin exposure substantially prevented lung fibrosis and collagen deposition. MEx treatment blunted inflammation and reduced classical (Ly6Chi CCR-2+ve) monocytes in the lung. Exploration of the upstream effects of MEx revealed that MEx induced a shift from classical to regulatory monocyte phenotype in the bone marrow. Interestingly, the adoptive transfer of MEx-pretreated BMDMo sufficed to alleviate fibrosis. Additionally, MEx prevented alveolar epithelial cell apoptosis.

Conclusion: It was shown that systemic therapy with MEx prevented fibrosis if administered during early or late stages of inflammation. It was further shown that MEx exert systemic immunomodulatory effects by regulation of monocyte phenotypes in the bone marrow that protected the lung from fibrosis. These results suggest the potential use of MEx for cell-free therapy in fibrotic lung diseases.

### Introduction

Idiopathic pulmonary fibrosis (IPF) is a chronic progressive respiratory disease with a prevalence of 0.5 to 27.9 per 100,000 person years (1, 2). The lack of complete understanding of the underlying mechanism of this disease, may have contributed to the paucity of successful therapies. Despite two newly approved drugs, IPF remains fatal with a five-year survival rate of less than 10% (3-6). In addition to pharmacologic therapy, cell-based therapies such as mesenchymal stromal cells (MSCs) have also been explored (7-9). Despite promising results with MSC therapy in the prevention of lung fibrosis, limitations such as adverse immune reactions, survival challenges, unexpected engraftments, potential for MSC-to-fibroblast differentiation, nevertheless, continue to render cell-free therapies highly desirable (8-10).

It was previously demonstrated that the therapeutic capacity of MSCs reside in their secretome, which is composed of a heterogeneous pool of bioactive molecules, often enclosed in extracellular vesicles (EVs). In pre-clinical models other than IPF, e.g. bronchopulmonary dysplasia, pulmonary hypertension and acute lung injury, EVs or more specifically exosomes (MEx) isolated from MSC secretome, have been shown to act as the therapeutic vector (7, 11-19).

The effect of MEx in IPF is unknown. A growing body of literature supports the role of circulating inflammatory monocytes and alveolar M2-like macrophages in the development and progression of pulmonary fibrosis (20, 21). Additionally, recent reports in bleomycin-induced fibrosis models suggest a detrimental role for monocyte-derived alveolar macrophages (AM) that populate the lung after lung injury (21, 22). Whether MEx have any systemic and immunomodulatory effects on monocytes remains unknown. Additionally, the source of action of MEx is yet to be defined.

In this study, it was shown that systemic therapy with purified MEx prevented pulmonary fibrosis if administered during early or late stages of inflammation (day 0 and 7 after the administration of bleomycin). It was further revealed the systemic and organ-level effects of MEx in the modulation of macrophage and monocyte phenotypes. It was demonstrated that MEx exert an anti-apoptotic and immunomodulatory effect by altering the monocyte subpopulation from an inflammatory to a regulatory phenotype in the bone marrow. The latter findings led to the discovery that even the systemic delivery (adoptive transfer) of MEx-treated bone marrow-derived monocytes (BMDMo) prevented lung fibrosis. This study provides mechanistic insights into the action of MEx, supporting a systemic immunomodulatory potential leading to secondary antifibrotic effects in the lung.

### Methods:

### Animal Models, Histology and Cytometry

All mice were housed and cared for in a pathogen-free facility. All animal experiments were approved by the Boston Children's Hospital Animal Core and Use Committee. Ten to fourteen-week-old C57BL/6 mice (Charles Laboratories) were anaesthetized with isoflurane and endotracheally injected with a dose of 3 U/kg of bleomycin sulfate in 50 μl of 0.9% normal saline (NS) or NS alone on day 0. Mice received 200 μl of bolus dose of MEx, (EVs produced by 5×10⁶ MSCs, treatment group), human dermal fibroblast-derived exosomes (FEx); (EVs produced by 5×10⁶ human dermal fibroblasts cells, first control group) or OptiPrep™ (iodixanol, IDX, 1:9 dilution); (vehicle, second control group) or NS via tail vein injection on days 0 and 7.

After bleomycin treatment at designed time points, mice were euthanized with intraperitoneal injection of pentobarbital. The hearts were perfused with phosphate-buffered saline (PBS, invitrogen) through the right ventricle.

For histologic analysis, trachea was cannulated and lungs were inflated with 4% paraformaldehyde. Right lung was embedded in paraffin and sectioned for hematoxylin and eosin or Masson's trichrome staining. The left lung was either snap frozen in liquid nitrogen and used for RNA and protein isolation or used fresh for collagen quantification or cytometric analysis. Randomly selected areas (10-15 fields) from 5 μm thick lung sections were acquired at ×100 and ×200 magnification using a Nikon Eclipse 80i microscope (Nikon, Tokyo, Japan). Large airways and vessels were not imaged. For histologic quantification, the Ashcroft score was used in a blinded fashion. Scores of 0-1 represented no fibrosis, scores of 2-3 represented minimal fibrosis, scores of 4-5 were considered as moderate fibrosis, and scores of 6-8 indicated severe fibrosis (23).

BMDMo were isolated as described previously (11). Cell suspension was used for cytometric analysis and cultured adherent cells after 3 days were used for adoptive transfer experiments (further details can be found in online supplementary material).

### Exosome Isolation and Purification

Exosome isolation, purification and characterization were performed as described previously using OptiPrep™ (iodixanol; IDX) cushion density flotation (11). Briefly, concentrated conditioned media from bone marrow MSCs or human dermal fibroblasts (HDFs) was floated on top of IDX cushion and centrifuged for 3.5 hours at 100,000×g at 4° C.

### Statistics

Data between different groups was compared using ANOVA with Fisher's LSD test post hoc analysis on GraphPad Prism (v6.0; GraphPad, CA, US). Flow cytometry data analyses were performed using FlowJo software v10.2 (TreeStar, OR, US). The mRNA levels were assessed by RT-qPCR and expressed relative to endogenous control. The ΔCT was used for statistical analysis. Data are presented as mean±standard error of mean (SEM). Significance was determined with respect to the p<0.05 threshold unless stated otherwise. A minimum of 5 animals were used in each group to yield >90% power at the 5% α-level.

### Results

### MEx Administration During Early Inflammation Prevents Lung Fibrosis

A well-established bleomycin lung injury model was used for pulmonary fibrosis characterized by an inflammatory (day 0 to 8) followed by a fibrotic stage (day 9 to 32) (24).

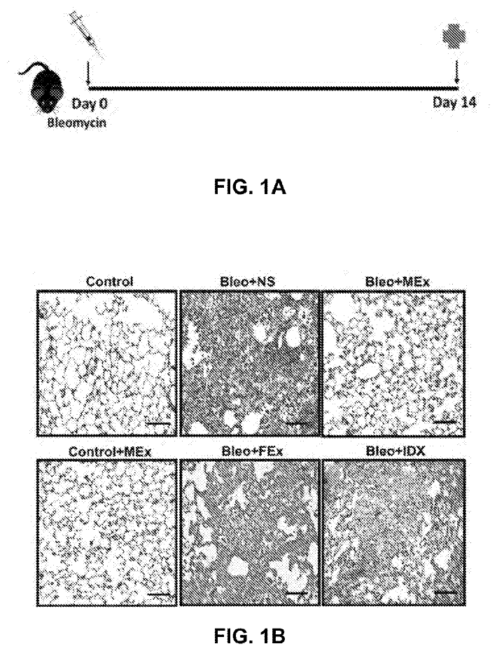

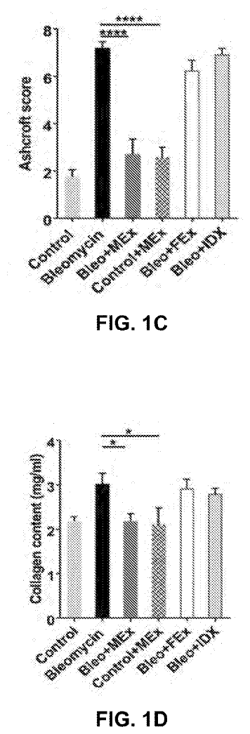

To investigate the role of MEx in the prevention of pulmonary fibrosis, ten to fourteen-week old mice received endotracheal bleomycin (3 U/kg) or NS (vehicle, control) on day 0 followed by a bolus dose of intravenous (IV) MEx via tail vein. Mice were sacrificed at day 14 and lungs were assessed for fibrosis quantification and collagen content (FIG. 1A). Bleomycin increased the Ashcroft score more than threefold compared to control mice. There was a significant reduction in fibrosis score in the MEx-treated mice (Bleo+MEx) compared to the bleomycin group (Bleo+NS, FIGS. 1B and 1C). Similarly, the increase in collagen deposition elicited by bleomycin was substantially reduced in Bleo+MEx mice (FIG. 1D). To ensure that the therapeutic effect is unique to MEx, bleomycin-exposed mice were injected with fibroblast exosomes (Bleo+FEx) and iodixanol (Bleo+IDX) as well. No amelioration in fibrosis or collagen deposition was seen in the aforementioned groups. To exclude the potential for lung architectural changes with MEx treatment, control mice were injected with MEx. The treatment was well tolerated in mice and lung collagen content and histology was similar to the control mice receiving NS (FIGS. 1B to 1D). These results show that a single dose of IV MEx at the beginning of the inflammatory phase prevents fibrosis. This effect is unique to MSC exosomes as exosomes derived from fibroblasts [and the exosome isolation medium (iodixanol)] did not prevent lung fibrosis.

### MEx Therapy at the End of Inflammation Reverts Lung Fibrosis

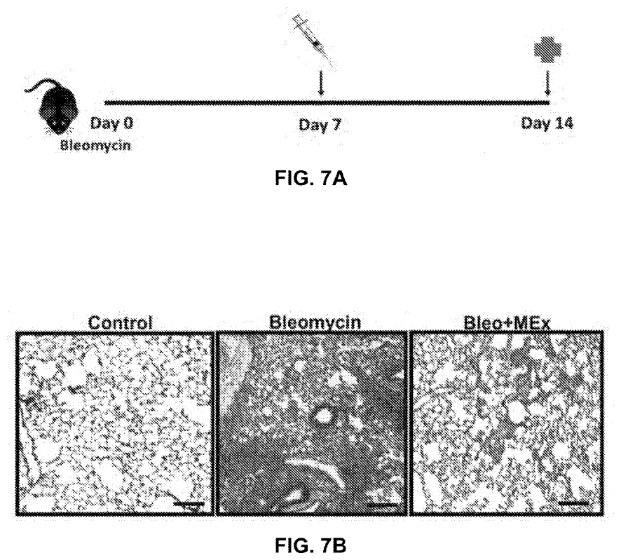

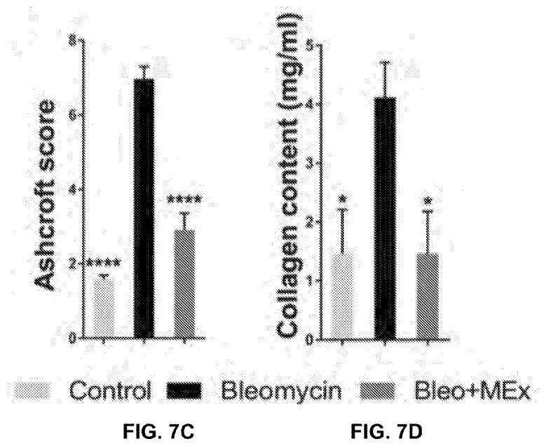

In order to assess the effect of MEx at later stages of inflammation, mice were injected with MEx 7 days after bleomycin administration (FIG. 7A). Similar to what was observed in the preventive therapy experiment (MEx injection on day 0), administration of MEx during the inflammatory stage led to an improvement in fibrosis scores and a statistically significant reduction in collagen deposition (FIGS. 7B, 7C and 7D). Therefore, MEx therapy ameliorates fibrosis even if administered at the end of inflammation.

### MEx Modulate Alveolar Macrophage Phenotypes and Blunt Inflammation

To investigate the mechanism of action of MEx in the bleomycin lung injury model, preventive therapy experiment (MEx injection on day 0) were carried out.

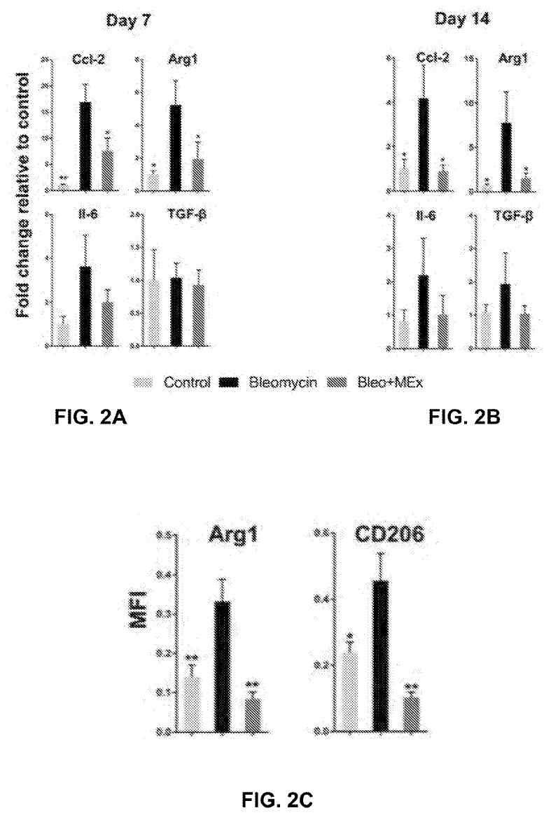

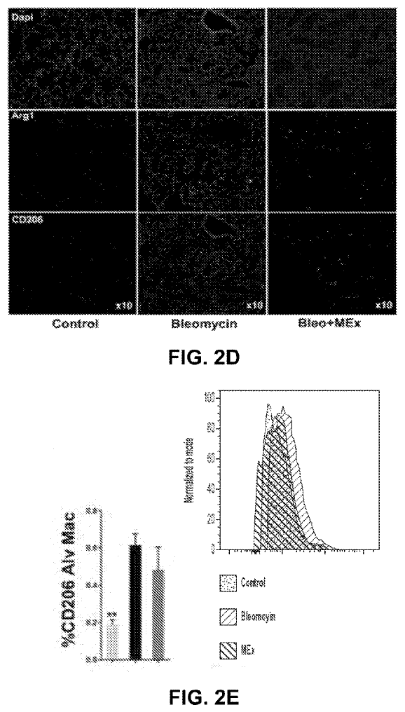

Monocyte-derived macrophages participate in the development and progression of fibrosis (20, 21), thus, the role of MEx was assessed in the modulation of inflammation through regulation of inflammatory and profibrotic macrophage phenotype. Gene expression analysis in whole lung 7 or 14 days after the administration of bleomycin, showed an increase in the expression levels of the macrophage inflammatory markers, Ccl-2 and Arginase-1 (Arg 1), while their mRNA levels were comparable to those observed in control lungs with MEx treatment. Interleukin-6 mRNA levels showed a similar trend to that of Cc1-2 and Arg1, though the difference did not reach statistical significance between groups. Moreover, TGF-β expression was similar at both time points in all three experimental groups (FIGS. 2A and 2B). Immunofluorescence (IF) staining of lung tissue sections with CD206 and Arg1 antibodies which are macrophage markers of M2-like activation, showed an increase in IF intensity in mice that received bleomycin but remained similar to control levels when mice were treated with MEx (FIGS. 2C and 2D). Flow cytometric analysis of whole lungs also showed an increase in CD206 expressing alveolar macrophages (AM) (CD45+veCD11b-veCD11c+veCD206+ve cells) in bleomycin mice. Despite lower number of CD206 expressing AMs with MEx treatment, the levels did not reach statistical significance (FIG. 2E). The above results reveal that MEx exert anti-inflammatory effects through the modulation of AM phenotype in the lung.

### MEx Restore Alveolar Macrophage and Regulatory Monocyte Population in the Lung

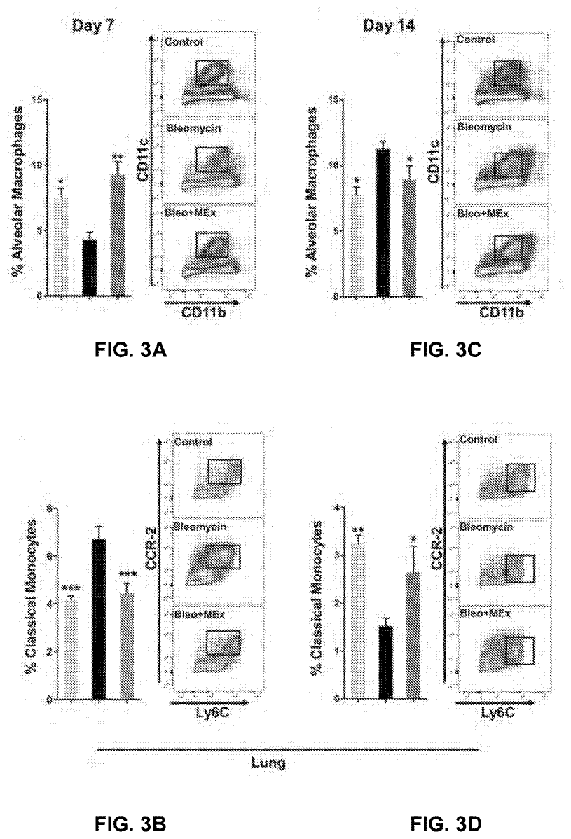

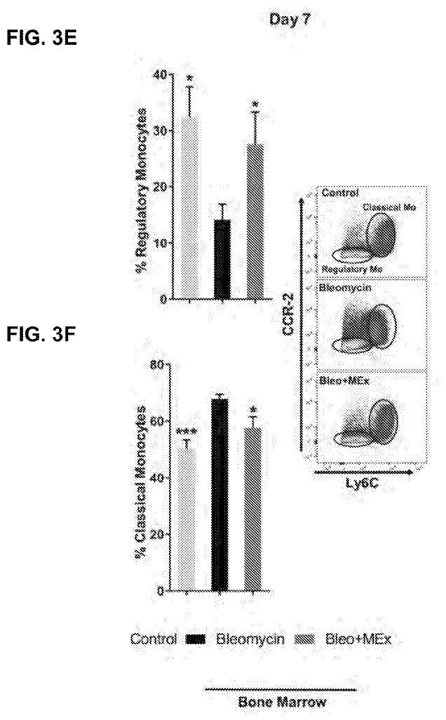

To investigate the dynamic changes in immune cell populations with bleomycin injury and after MEx therapy, cytometric analysis on whole lungs was performed at days 7 or 14 following the administration of bleomycin. A decrease in AM numbers (CD45+veCD11b-veCD11c+ve cells) was noted in bleomycin-treated mice on day 7 (FIG. 3A). This was associated with an increase in the number of Ly6Chi classical or inflammatory monocytes (CD45+veCD11b+veMHC II-veLy6ChiCCR-2+ve cells) (FIG. 3B). On day 14 however, the proportion of AMs after bleomycin instillation was increased (FIG. 3C) while the number of classical monocytes was reduced (FIG. 3D). MEx therapy led to the restoration of the AMs and infiltrating monocyte populations to levels similar to control group both at day 7 and 14. These results show that following lung injury, MEx can restore the homeostatic balance between AM and recruited monocyte populations to similar to levels and phenotypes found in control mice.

### MEx Can Modulate Monocyte Phenotypes in the Bone Marrow

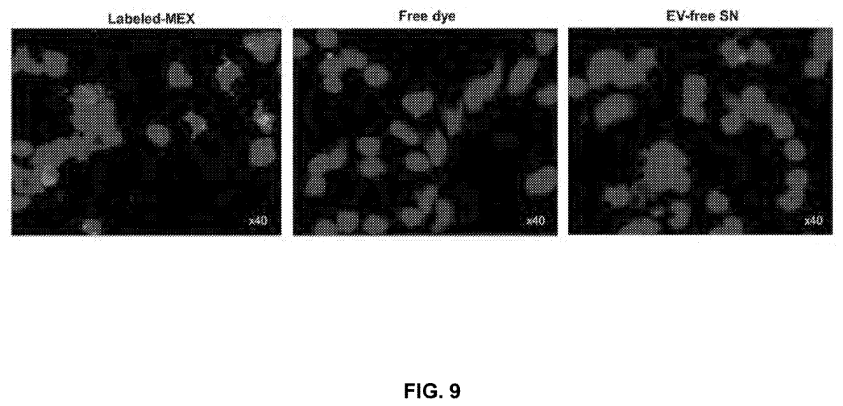

Following the observation of increased inflammatory monocytes in the lungs of bleomycin-exposed mice, and the restoration to normal levels after MEx therapy, and given the fact that monocyte development occurs in the bone marrow (BM) (25) it was proposed that MEx may exert immunomodulatory effects by modifying the monocyte phenotypes in the BM. To answer this question, the potential of MEx was first investigated to infiltrate the BM. Dye labeled-EVs were IV injected into control mice and the animals were sacrificed at 2, 4, 8 and 24 hours after injection. Dapi staining of BM cytospins revealed the presence of EVs in the BM up to 8 hours after injection (FIG. 9, images represent 2 hours after injections, further time points not shown).

The systemic effects of MEx was subsequently researched by looking at the signature of myeloid cells in the BM. Interestingly, flow cytometric analysis of myeloid cells isolated from the BM of control, bleomycin, and MEx-treated mice during the active inflammatory phase (day 7) showed similar changes to what was observed in the lung. Despite comparable numbers of CD45+ve cells obtained in the three experimental groups (data not shown), regulatory monocyte number (Cd45+veCD11bhighMHC II-veLy6ClowCCR-2-ve cells) was less than half in bleomycin-exposed mice compared to MEx-treated and control mice (14.18% vs 27.57% and 32.3±5.7 respectively, FIG. 3E). In contrast, the monocyte population in the bleomycin-exposed group consisted of ˜70% (67.8%±1.7) classical monocytes compared to approximately 50-60% in the MEx-treated and control group of mice (57.5%±3.9 and 50.1%±3.2 respectively, FIG. 3F). These results suggest that in the presence of organ injury, MEx exert immunomodulatory effects by the alteration of monocyte populations from a pro-inflammatory to a regulatory phenotype in the bone marrow.

### The Immunomodulatory Influence of MEx on BMDMo Suffices to Prevent Pulmonary Fibrosis

Given the increase in BM regulatory monocytes after MEx therapy, it was hypothesized that the immunomodulatory effects of MEx on bone marrow monocytes might suffice to prevent fibrosis, and that further changes in the lung are the consequence of an altered BM monocyte subpopulations.

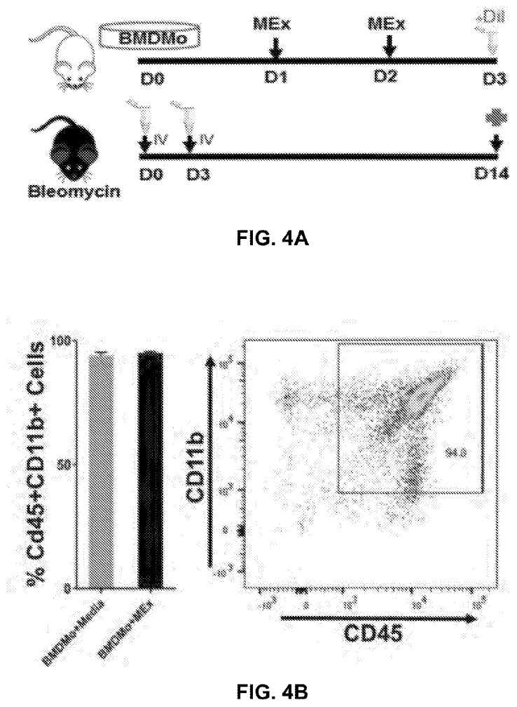

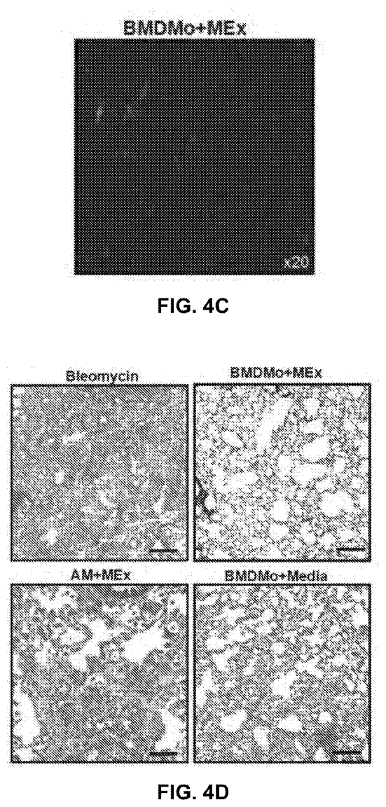

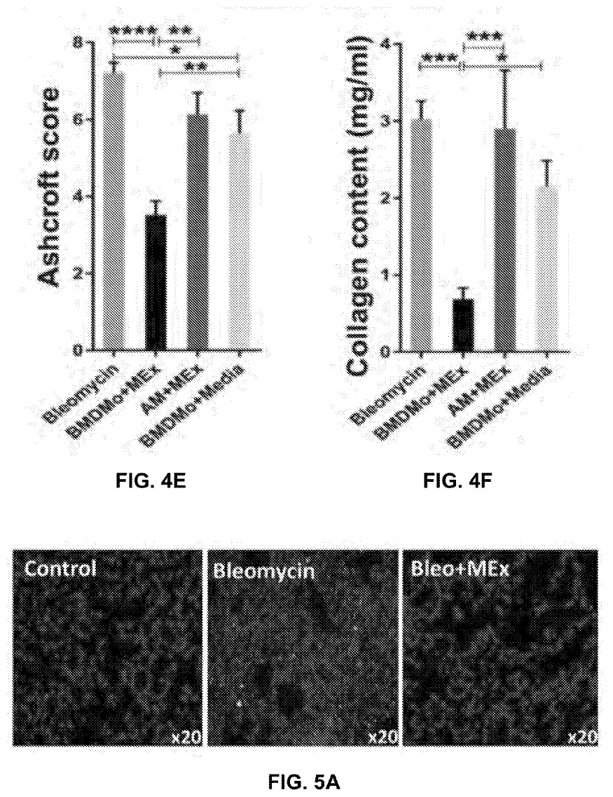

To test this hypothesis, the effect of ex vivo treated BMDMo was explored in the prevention of fibrosis. Adoptive transfer experiments were performed in which primary Mos were isolated from wild type FVB mice and cultured for 3 days. Cells were treated with MEx (BMDMo+MEx) or media alone (BMDMo+Media) on days 1 and 2 (FIG. 4A). On day 3 it was confirmed that more than 90% of the bone marrow cells were CD45+veCD11b+ve (myeloid subset, FIG. 4B). Monocytes were then labeled with Dil (fluorescent lipophilic dye) and adoptively-transferred intravenously to C57BL/6 mice at day 0 and 3 after instillation of bleomycin. Mice were sacrificed at day 14 and lungs were assessed for histology and collagen content. Results were compared to mice that received bleomycin with NS injection (bleomycin). The Dil-labeled monocytes were identified in the lungs 14 days after the administration of bleomycin (FIG. 4C). Interestingly, less fibrosis was detected both with histologic quantification and collagen assay in mice that received BMDMo+MEx compared to bleomycin and BMDMo+Media-receiving mice. Surprisingly, minimal amelioration of fibrosis score on histology and statistically non-significant collagen deposition in the BMDMo+Media -treated group compared to bleomycin-exposed mice (FIGS. 4D, 4E, and 4F) were detected. To investigate if the anti-fibrotic effects may be due to resident AMs instead, MEx-treated AMs (AM+MEx) were administered endotracheally following bleomycin instillation (details are described in supplementary methods). Any amelioration of fibrosis was not detected in mice who received pretreated AMs compared to the bleomycin group (FIG. 4E).

These data strongly suggest that treatment of BMDMo with MEx promote a regulatory phenotype that by itself ameliorates fibrosis. This further confirms that the therapeutic influences of MEx are not confined to the lung and that MEx exert systemic anti-inflammatory effects by modulating the bone marrow monocytic phenotype which leads to the dampening of inflammation and prevention of fibrosis in the injured lung.

### MEx Therapy Decreases Apoptosis

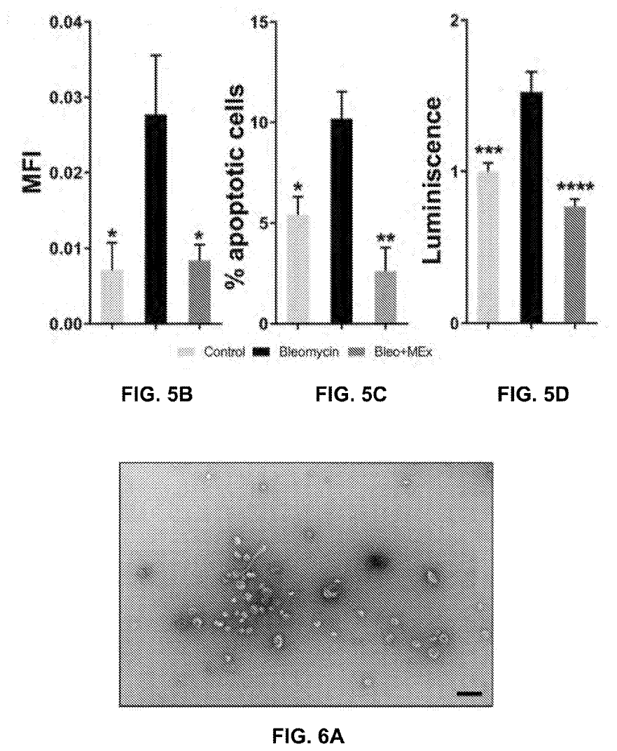

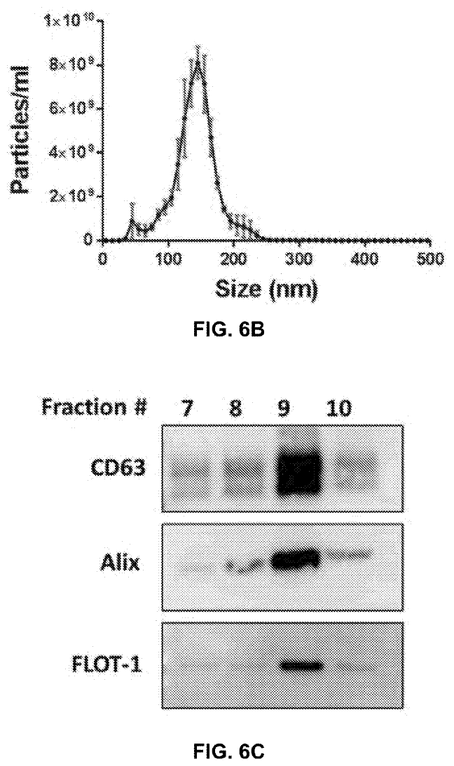

Alveolar epithelial cell apoptosis (AEC) has been described as a trigger for a pro-fibrotic signal in damaged lungs (26, 27). To explore further mechanisms by which MEx protect from lung injury, the potential role of MEx in the reduction of apoptosis following bleomycin injury was investigated. The degree of lung apoptosis was assessed using tunel staining on lung sections from control, bleomycin, and MEx-treated mice. There was an increase in apoptosis noted in the bleomycin-exposed group, while apoptosis levels were similar in Bleo+MEx and control mice (FIG. 5A, 5B). Additionally, Annexin V/PI staining in whole lungs at day 14 was performed. There was again an increase in apoptosis (Annexin V+/PI−) present in bleomycin compared to control and MEx-treated mice (FIG. 5C).

Furthermore, the direct anti-apoptotic effect of MEx on human alveolar epithelial cells (A549, AEC) was assessed. An in vitro assay was designed where epithelial cell apoptosis was induced by treating A549 cells with bleomycin. A group of bleomycin-exposed AECs were treated with MEx for 24 hours and changes in apoptosis were determined by caspase 3 and 7 activity using Caspase-Glo® 3/7 luminescence assay. An increase in apoptosis in the bleomycin group was noted which was abrogated in MEx-treated cells (FIG. 5D). The above findings support an important anti-fibrotic effect of MEx in vitro and in vivo.

### Discussion

This study shows that a single IV dose of human bone marrow-derived MEx either at the induction or at the end of the inflammatory phase of bleomycin-induced lung injury strikingly prevents fibrosis and restores lung architecture. MEx treatment not only blunted inflammation in the lung, but also restored AMs and recruited monocytes numbers to levels similar to control mice. The aforementioned observation and the fact that monocyte development stems in the bone marrow (BM), led to the investigation of the upstream immunomodulatory effects of MEx by researching the BM myeloid signature. In addition to visualization of labeled-MEx in the BM, flow cytometric analysis of BM myeloid cells revealed a shift in monocyte subpopulation from a pro-inflammatory (Ly6ChiCCR-2+ve) to a regulatory (Ly6ClowCCR-2-ve) phenotype in MEx-treated mice.

Interestingly, using novel MEx-pretreated BMDMo adoptive transfer experiments it was shown that the immunomodulatory effects of MEx on the BM monocytes at least partly suffice to explain their protective effect in the lung. Finally, other potential mechanisms in the protection against lung fibrosis were explored and noted a decrease in apoptosis in the lungs of MEx-treated mice. Furthermore, the in vitro experiments revealed that MEx exert anti-apoptotic effects by targeting the alveolar epithelial cell.

To rationalize the cytometric results at different time points (day 7 and 14) in bleomycin-exposed mice, previous findings were considered that recruited inflammatory (Ly6Chi) monocytes and monocyte-derived alternatively-activated macrophages (M2-like) were associated with the development and progression of fibrosis (20-22, 28-31). Additionally, these results revealed that the increase in inflammatory monocytes following bleomycin lung injury originates in the BM. It is plausible that bleomycin-induced loss of resident AMs signals the BM stem cells to increase differentiation to pro-inflammatory monocytes, and these cells then populate the lungs during the inflammatory phase (as seen on day 7 in the model). These classical monocytes differentiate into M2-like AMs at later stages of injury and provide a profibrotic milieu that further exacerbates the fibrotic response. This explains the increase in AMs and their inflammatory markers on day 14.

MSC-EVs can repopulate Sca-1 positive and c-kit low-positive stem cells in the BM of irradiated mice (32). They have also been shown to modulate monocytes trafficking in a model of myocarditis (33). In the presence of organ injury, MSC-EVs may reprogram myeloid stem cells to differentiate into a regulatory phenotype. Accordingly, there was an increase in regulatory monocytes in the BM and a reduction in inflammatory monocytes in the lung, and therefore, less differentiation to profibrotic macrophages. Prevention of fibrosis with the adoptive transfer of MEx-treated BMDMo strongly suggests that the alteration of BM monocyte phenotype is a mechanistic explanation for the subsequent anti-fibrotic effect of MEx in the lung. This effect was not recapitulated with endotracheal injection of MEx-treated AMs. In a recent study by Morrison and colleagues the endotracheal administration of MSC-EV-treated AMs to an LPS-induced acute lung injury model, decreased inflammation (17). While these results also agree with the immunomodulatory effect of MSC-EV on macrophages, lack of improvement in fibrosis after AM transfer in this experiment can be due to the differences in disease models and therefore different underlying pathophysiology. Van de Laar and colleagues demonstrated that both mature AMs and BMDMo have the capacity to colonize an empty AM niche and develop into functional tissue-resident macrophages (34). It is possible that the absence of an empty AM niche at the beginning of inflammation (day 0 to 3 in the adoptive transfer experiment) did not allow sufficient colonization by the transplanted AMs.

Finally, using different in vivo methods, it was shown that in addition to immunomodulation, MEx could also potentially prevent fibrosis through the reduction of apoptosis. Furthermore, the in vitro assay described herein suggests that this effect is produced by targeting the alveolar epithelial cells.

There are limitations to this study. The current therapeutic dose was estimated based on the previous experiments. Thus, future studies should be performed to investigate dose responses.

This study investigated the effects of MSC exosomes in an experimental model of IPF. The findings provide new insights into the systemic inflammatory responses following bleomycin lung injury and the alterations in monocyte phenotypes in the bone marrow. Additionally, this study uncovers new mechanistic explanations for the immunomodulatory effects of MSC exosomes and their source of action. MSC exosomes are believed to be a promising cell-free therapy for the treatment of fibrotic lung diseases if administered early in the course of disease.

### Supplemental Methods

### Cell Isolation and Culture

Human bone marrow mesenchymal stem cells (BMSCs) were obtained from RoosterBio (RoosterBio, MD, US). Human foreskin (dermal) fibroblast cells (HDFs) were established by tissue explant method (36). BMSCs and HDFs were cultured and expanded and further characterized as described previously (37). A549 Alveolar epithelial cells (ATCC) were cultured in F-12K medium (Thermo Fisher Scientific, Inc., Waltham, Mass.).

### Transmission Electron Microscopy (TEM)

An aliquot of 5-10 μl of extracellular vesicle (EV) preparation was adsorbed for 15 seconds on a formvar/carbon coated grid (Electron Microscopy Sciences, PA, US). Samples were stained with 2% uranyl acetate after removal of excess liquid with Whatman Grade 1 filter paper (Sigma). EVs were then viewed by a JEOL 1200EX transmission electron microscope (TEM), and images were recorded with an AMT 2k CCD camera.

### Nanoparticle Tracking Analysis

Size and concentration distributions of exosomes were determined using nanoparticle tracking analysis (NTA, NanoSight LM10 system, Malvern instruments, MA, US) as described previously (37).

### Western Blot Analysis

Proteins in exosome preparations were separated on a 4-20% polyacrylamide gel (Bio-Rad, Hercules, Calif.), followed by transfer to 0.45 μm PVDF membrane (Millipore, Mass., US). Rabbit polyclonal anti-flotillin-1 and anti-CD63 antibodies (Santa Cruz Biotech, Calif., US), and mouse monoclonal anti-Alix antibody (Santa Cruz Biotech, Calif., US) were used based on recommended dilutions by the manufacturer.

### EV Dosing

EV preparations were diluted on PBS to correspond to 5×10⁶ cell equivalent. This dose was estimated based on previous dose calculation in newborn mice with corresponding NTA and protein concentrations (37).

### Immunofluorescence Staining

Lung tissue sections were de-paraffinized in xylene and rehydrated. Tissue slides were treated with 10 mM citrate buffer and blocked with serum and BSA for 20 min. Samples were then incubated at 40 C overnight with indicated primary antibody, Arginase 1 (Santa Cruz Biotech, Calif., US); CD206 (Santa Cruz Biotech, Calif., US), then further incubated with secondary antibody (Life technologies, MA, US) for 20 minutes followed by nuclear staining with DAPI for 10 minutes.

Arginase 1 and CD206 positive cells were imaged using a Nikon Eclipse 80i microscope (Nikon, Tokyo, Japan). 10-15 random images were analyzed using image J software.

Mean Fluorescence Intensity (MFI) was calculated using the following formula: MFI=Integrated Density−(Area of selected cell*Mean fluorescence of background reading).

### Sircol Collagen Assay

The left lung was used for collagen quantification per manufacturer protocol (Biocolor, Life Science Assays). Briefly, left lung homogenate were shaken overnight at 4° in 5 ml of 0.5 M acetic acid with 0.6% pepsin. One ml of dye reagent was added to 100 μl of transparent pernatant and the samples were vortexed for 30 minutes. The residual pellet was washed by acid-salt wash buffer to eliminate unbound collagen and pH was normalized with alkalization buffer. Absorbance was measured at a wavelength of 550 nm in a microplate reader. Measured collagen content was compared to a standard curve and represented as mg/ml of left lung homogenate.

### Cytometric Analysis of Mouse Whole Lung and Bone Marrow

Lung macrophage populations were assessed by flow cytometry as previously described (38). Lungs were harvested on days 7 and 14. Left lung was cut into small pieces and digested in 5 ml of digestion buffer consisting of RPMI-1640 (Invitrogen, CA, US), Collagenase IV (1.6 mg/ml); and DNAse1 (50 unit/ml), both from Worthington Biochemical Corp, NJ, US. Lung were shaken at 37° C. for 30 minutes and red blood cells (RBC) were lysed using RBC lysis buffer (Roche, Ind., US). Homogenized lung was passed through a 40 μm cell strainer (Corning, Mass., US) to obtain a single-cell suspension.

For the assessment of alveolar macrophage and monocyte populations, the cell suspension was stained with antibodies; PE/Cy7-conjugated anti-mouse CD45, FITC-conjugated anti-mouse CD11b, PerCP Cy 5.5-conjugated anti-mouse CD11c, BV 421-conjugated anti-mouse CD206, BV 605-conjugated anti-mouse MHC II, BV 510-conjugated anti-mouse Ly6C and Alexa 647-conjugated anti-mouse CCR-2.

For the evaluation of bone marrow derived monocytes (BMDMo), freshly flushed cells from the femur and tibia of adult mice were stained with PE/Cy7-conjugated anti-mouse CD45, FITC-conjugated anti-mouse CD11b, BV 605-conjugated anti-mouse MHC II, BV 510-conjugated anti-mouse Ly6C and Alexa 647-conjugated anti-mouse CCR-2 (all antibodies were obtained from Biolegend, CA, US). Similar staining was performed on harvested BMDMo after 3 hours of in vitro culture.

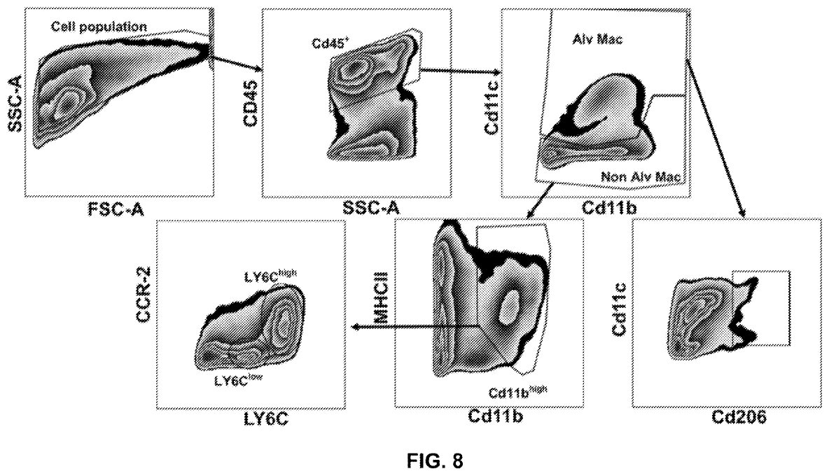

Compensation was adjusted accordingly and supported by UltraComp ebeads (Affymetrix, CA, US). Fluorescence-minus-one controls were used accordingly. Cell populations were identified according to the gating strategy illustrated in FIG. 8 and recorded as a percentage of total cell population.

### Reverse Transcription-Polymerase Chain Reaction Analysis

Total RNA was extracted from left lung using TRIZOL® (Thermo Fisher Scientific, Inc., Waltham, Mass.) as per manufacturer's instructions. TaqMan® primers used in the PCR reactions including Cc12, 116, TGF-β, and Arginase 1 were obtained from Invitrogen. Nuclear pore protein 133 served as an internal control. Analysis of the fold change was performed as previously described compared to control mice (39).

### Annexin V/PI Apoptosis Assay, Tunnel Staining and Caspase 3/7 Assay

Annexin V staining kit (Sigma-Aldrich, MO, US) was used to assess apoptosis in the whole lung. Single cell suspension was obtained from left lung as described above. Cells were then floated in 1× binding buffer and stained with FITC conjugated-Annexin V and PI antibody for 10 minutes and immediately assessed by flow cytometry. Apoptosis was assessed in paraffin-embedded lung tissue using TACS® TdT in situ—Fluorescein tunnel assay (R&D systems, MN, US) per manufacturer protocol. Briefly, deparaffinized lung sections were permeabilized using Cytonin for 1 hour and labeled with a combination of Mangenese cation, TdT dNTP Mix, and TdT enzyme followed by incubation with Strep-Fluor solution for 20 minutes. Fluorescent imaging and quantification was performed as described above.

Caspase 3/7 assays (G8090, Promega) were performed according to the manufacturer's instructions. Briefly, 2×10⁴ A549 alveolar epithelial cells were plated overnight in a 96-well plate. Cells were treated with 0.1 μg/well of bleomycin sulfate or media alone for 24 hours (8 wells per group). This was followed by treatment of the bleomycin-treated cells with 10/well of MEx (equivalent to EVs produced by approximately 2×10⁴ MSCs) for 24 hours. Bleomycin-treated cells treated with media only were used as control. All the experiments were performed in serum free medium. On day 3, cells were washed with PBS and 50 μl of fresh media was added to each well. To measure caspase 3/7 activity, 50 μl of caspase Glo 3/7 reagent was added to each well for 2 h at room temperature and the plate was left on a plate shaker. Luminescence was measured using VICTOR Multilabel plate reader. The background luminescence (measured in cell-free well) was subtracted from each read-out.

### Adoptive Transfer of MEx Treated Bone Marrow Derived Monocytes

BMDMo were isolated from 6-8 wk-old FVB by flushing the femur and tibia and culturing cells for 3 days in Dulbecco's Modified Eagle Medium (DMEM) supplemented with 10% FBS, containing 30% v/v L929-conditioned medium (as a source of macrophage colony-stimulating factor; M-CSF). Each plate was treated with MEx generated from 1×10⁶ MSCs or media only on days 1 and 2. Cells were harvested on day 3 and after two washes with PBS, stained with Dil as per the manufacturer protocol (Life technologies). BMDMo were then administered via tail vein injection at a 1:1 ratio (BMDMo isolated from one mice were injected into the experiment mouse) on day 0 and day 3 after endotracheal instillation of bleomycin.

### Adoptive Transfer of MEx Treated Murine Derived Alveolar Macrophages

Six to eight-weeks FVB mice were euthanized by i.p. pentobarbital injection. The anterior wall of the trachea was cannulated with a 21-gauge needle and secured using a string. Bronchoalveolar lavage fluid (BALF) was collected with 5 flushes of 0.6 ml of sterile HBSS (supplemented with 0.5 mM EDTA and 1 mM HEPES) using a 1 ml syringe. BALF was centrifuged at 400×g for 5 min and the supernatant was aspirated. Murine AMs were resuspended in fresh RPMI media supplemented with 1% penicillin/streptomycin and 10% FBS and were seeded in a 35 mm plate at a seeding density of 1×10⁶ per plate. Each plate was treated overnight with MEx generated from 1×10⁶ cells. The cells were harvested after 24 hours, washed twice with PBS, stained with Dil and re-suspended in 50 μl of PBS. AMs were administered endotracheally at a one-to-one (AMs isolated from one mouse were administered to the experiment mouse) ratio on day 0 and 3 following instillation of bleomycin.

### Ex Vivo EV Labelling and Bone Marrow Cytospins

EVs were pelleted for 70 minutes at 100,000 g from concentrated conditioned media of bone marrow MSCs. EV protein concentration was determined using micro BCA protein assay kit (Thermo Fisher Scientific, Inc., Waltham, Mass.). EVs were labeled by ExoGlow-Membrane™ EV Labeling Kit (System biosciences, CA, USA) per manufacture protocol. Briefly, 50-100 m of EVs were added to the mixture of reaction buffer and labeling dye and incubated at room temperature for 30 minutes. Free unlabeled dye was removed following a second ultracentrifugation at 100,000 g for 70 minutes. The EVs produced by equivalent of 1×10⁶ MSCs were diluted in 200 μl of PBS and injected into C57BL/6 mice using tail vein injection. 200 μl of stained EV-free SN, or diluted free dye were used as controls.

Mice were sacrificed at 2, 4, 8 and 24 hours following injections. The femur bones were flushed with PBS and cell suspension was cytocentrifuged at 300 g for 5 min using the Shandon Cytospin 4 (Thermo Fisher Scientific, Inc., Waltham, Mass.). Slides were air-dried, fixed with 4% paraformaldehyde and counterstained with Dapi. Images were obtained using a Nikon Eclipse 80i microscope (Nikon, Tokyo, Japan).

### REFERENCES

1. Kaunisto J, Salomaa E-R, Hodgson U, Kaarteenaho R, Myllarniemi M. Idiopathic pulmonary fibrosis—a systematic review on methodology for the collection of epidemiological data. BMC Pulmonary Medicine. 2013; 13:1.
2. Ley B, Collard H R. Epidemiology of idiopathic pulmonary fibrosis. Clin Epidemiol. 2013; 5:483-92.
3. Mylla M, Kaarteenaho R. Pharmacological treatment of idiopathic pulmonary fibrosis A preclinical and clinical studies of pirfenidone, nintedanib, and N-acetylcysteine. European Clinical Respiratory Journal. 2015; 2:1-10.
4. Harari S, Caminati A, Madotto F, Conti S, Cesana G. Epidemiology, survival, incidence and prevalence of idiopathic pulmonary fibrosis in the USA and Canada. Eur Respir J. 2017; 49(1).
5. Cottin V. The role of pirfenidone in the treatment of idiopathic pulmonary fibrosis. Respiratory research. 2013; 14 Suppl 1:S5.
6. Noble P W, Albera C, Bradford W Z, Costabel U, Glassberg M K, Kardatzke D, et al. Pirfenidone in patients with idiopathic pulmonary fibrosis (CAPACITY): two randomised trials. Lancet. 2011; 377(9779):1760-9.
7. Matthay M A, Anversa P, Bhattacharya J, Burnett B K, Chapman H A, Hare J M, et al. Cell Therapy for Lung Diseases. Report from an NIH-NHLBI Workshop, Nov. 13-14, 2012. American Journal of Respiratory and Critical Care Medicine 2013. p. 370-5.
8. Ghadiri M, Young P M, Traini D. Cell-based therapies for the treatment of idiopathic pulmonary fibrosis (IPF) disease. Expert Opinion on Biological Therapy. 2016; 16:375-87.
9. Toonkel R L, Hare J M, Matthay M A, Glassberg M K. Mesenchymal stem cells and idiopathic pulmonary fibrosis potential for clinical testing. American Journal of Respiratory and Critical Care Medicine. 2013; 188:133-40.
10. Srour N, Thebaud B. Mesenchymal Stromal Cells in Animal Bleomycin Pulmonary Fibrosis Models: A Systematic Review. Stem Cells Transl Med. 2015; 4(12):1500-10.
11. Willis G R, Fernandez-Gonzalez A, Anastas J, Vitali S H, Liu X, Ericsson M, et al. Mesenchymal Stromal Cell Exosomes Ameliorate Experimental Bronchopulmonary Dysplasia and Restore Lung Function through Macrophage Immunomodulation. Am J Respir Crit Care Med. 2018; 197(1):104-16.
12. Willis G R, Kourembanas S, Mitsialis S A. Toward Exosome-Based Therapeutics: Isolation, Heterogeneity, and Fit-for-Purpose Potency. Front Cardiovasc Med. 2017; 4:63.
13. Cruz F F, Borg Z D, Goodwin M, Sokocevic D, Wagner D E, Coffey A, et al. Systemic Administration of Human Bone Marrow-Derived Mesenchymal Stromal Cell Extracellular Vesicles Ameliorates Aspergillus Hyphal Extract-Induced Allergic Airway Inflammation in Immunocompetent Mice. Stem cells translational medicine. 2015; 4:1302-16.
14. Lee C, Mitsialis S A, Aslam M, Vitali S H, Vergadi E, Konstantinou G, et al. Exosomes mediate the cytoprotective action of mesenchymal stromal cells on hypoxia-induced pulmonary hypertension. Circulation. 2012; 126(22):2601-11.
15. Sdrimas K, Kourembanas S. MSC Microvesicles for the Treatment of Lung Disease: A New Paradigm for Cell-Free Therapy. Antioxidants & redox signaling. 2014; 21:1905-15.
16. Heldring N, Mager I, Wood M, Le Blanc K, El Andaloussi S. Therapeutic potential of multipotent mesenchymal stromal cells and their extracellular vesicles. Human gene therapy. 2015; 26:506-17.
17. Morrison T J, Jackson M V, Cunningham E K, Kissenpfennig A, McAuley D F, O'Kane C M, et al. Mesenchymal Stromal Cells Modulate Macrophages in Clinically Relevant Lung Injury Models by Extracellular Vesicle Mitochondrial Transfer. Am J Respir Crit Care Med. 2017; 196(10):1275-86.
18. Phinney D G, Di Giuseppe M, Njah J, Sala E, Shiva S, St Croix C M, et al. Mesenchymal stem cells use extracellular vesicles to outsource mitophagy and shuttle microRNAs. Nat Commun. 2015; 6:8472.
19. Burrello J, Monticone S, Gai C, Gomez Y, Kholia S, Camussi G. Stem Cell-1. Burrello, J. et al. Stem Cell-Derived Extracellular Vesicles and Immune-Modulation. Front. Cell Dev. Biol. 4, 1-10 (2016).Derived Extracellular Vesicles and Immune-Modulation. Frontiers in Cell and Developmental Biology. 2016; 4:1-10.
20. Gibbons M A, MacKinnon A C, Ramachandran P, Dhaliwal K, Duffin R, Phythian-Adams A T, et al. Ly6Chi monocytes direct alternatively activated profibrotic macrophage regulation of lung fibrosis. American Journal of Respiratory and Critical Care Medicine. 2011; 184:569-81.
21. Misharin A V, Morales-Nebreda L, Reyfman P A, Cuda C M, Walter J M, McQuattie-Pimentel A C, et al. Monocyte-derived alveolar macrophages drive lung fibrosis and persist in the lung over the life span. The Journal of experimental medicine. 2017; 214:2387-404.
22. McCubbrey A L, Barthel L, Mohning M P, Redente E F, Mould K J, Thomas S M, et al. Deletion of c-FLIP from CD11b(hi) Macrophages Prevents Development of Bleomycin-induced Lung Fibrosis. Am J Respir Cell Mol Biol. 2018; 58(1):66-78.
23. Hubner R H, Gitter W, El Mokhtari N E, Mathiak M, Both M, Bolte H, et al. Standardized quantification of pulmonary fibrosis in histological samples. Biotechniques. 2008; 44(4):507-11, 14-7.
24. Moeller A, Ask K, Warburton D, Gauldie J, Kolb M. The bleomycin animal model: a useful tool to investigate treatment options for idiopathic pulmonary fibrosis? Int J Biochem Cell Biol. 2008; 40(3):362-82.
25. Zimmermann H W, Trautwein C, Tacke F. Functional role of monocytes and macrophages for the inflammatory response in acute liver injury. Front Physiol. 2012; 3:56.
26. Cheresh P, Kim S J, Tulasiram S, Kamp D W. Oxidative stress and pulmonary fibrosis. Biochim Biophys Acta. 2013; 1832(7):1028-40.
27. Kim S J, Cheresh P, Jablonski R P, Williams D B, Kamp D W. The Role of Mitochondrial DNA in Mediating Alveolar Epithelial Cell Apoptosis and Pulmonary Fibrosis. Int J Mol Sci. 2015; 16(9):21486-519.
28. Bellon T, Martinez V, Lucendo B, del Peso G, Castro M J, Aroeira L S, et al. Alternative activation of macrophages in human peritoneum: implications for peritoneal fibrosis. Nephrol Dial Transplant. 2011; 26(9):2995-3005.
29. Braga T T, Agudelo J S H, Camara N O S. Macrophages during the fibrotic process: M2 as friend and foe. Frontiers in Immunology. 2015; 6:1-8.
30. Kolahian S, Fernandez I E, Eickelberg O, Hartl D. Immune Mechanisms in Pulmonary Fibrosis. 2016; 55:309-22.
31. Mora A L, Torres-Gonzalez E, Rojas M, Corredor C, Ritzenthaler J, Xu J, et al. Activation of alveolar macrophages via the alternative pathway in herpesvirus-induced lung fibrosis. Am J Respir Cell Mol Biol. 2006; 35(4):466-73.
32. Schoefinius J S, Brunswig-Spickenheier B, Speiseder T, Krebs S, Just U, Lange
33. C. Mesenchymal Stromal Cell-Derived Extracellular Vesicles Provide Long-Term Survival After Total Body Irradiation Without Additional Hematopoietic Stem Cell Support. Stem Cells. 2017; 35(12):2379-89.
34. Miteva K, Pappritz K, El-Shafeey M, Dong F, Ringe J, Tschope C, et al. Mesenchymal Stromal Cells Modulate Monocytes Trafficking in Coxsackievirus B3-Induced Myocarditis. Stem Cells Transl Med. 2017; 6(4):1249-61.
35. van de Laar L, Saelens W, De Prijck S, Martens L, Scott C L, Van Isterdael G, et al. Yolk Sac Macrophages, Fetal Liver, and Adult Monocytes Can Colonize an Empty Niche and Develop into Functional Tissue-Resident Macrophages. Immunity. 2016; 44:755-68.
36. Nichols W W, Murphy D G, Cristofalo V J, Toji L H, Greene A E, Dwight S A. Characterization of a new human diploid cell strain, IMR-90. Science. 1977; 196(4285):60-3.
37. Willis G R, Fernandez-Gonzalez A, Anastas J, Vitali S H, Liu X, Ericsson M, et al. Mesenchymal Stromal Cell Exosomes Ameliorate Experimental Bronchopulmonary Dysplasia and Restore Lung Function through Macrophage Immunomodulation. Am J Respir Crit Care Med. 2018; 197(1):104-16.
38. Misharin A V, Morales-Nebreda L, Mutlu G M, Budinger G R, Perlman H. Flow cytometric analysis of macrophages and dendritic cell subsets in the mouse lung. Am J Respir Cell Mol Biol. 2013; 49(4):503-10.
39. Lee C, Mitsialis S A, Aslam M, Vitali S H, Vergadi E, Konstantinou G, et al. Exosomes mediate the cytoprotective action of mesenchymal stromal cells on hypoxia-induced pulmonary hypertension. Circulation. 2012; 126(22):2601-11.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### Equivalents and Scope

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of regulating a monocyte phenotype, the method comprising contacting a monocyte with an isolated mesenchymal stem cell (MSC) exosome.

2. The method of claim 1, wherein the monocyte is from bone marrow.

3. The method of claim 1 or claim 2, wherein the isolated MSC exosome is isolated from MSC-conditioned media.

4. The method of any one of claims 1-3, wherein the MSC is from Wharton's Jelly, bone marrow, or adipose tissue.

5. The method of any one of claims 1-4, wherein the isolated MSC exosome is substantially free of protein contaminants.

6. The method of any one claims 1-5, wherein the isolated MSC exosome has a diameter of about 50-150 nm.

7. The method of any one of claims 1-6, wherein the contacting is in vitro.

8. The method of any one of claims 1-6, wherein the contacting is ex vivo.

9. The method of any one of claims 1-6, wherein the contacting is in vivo.

10. The method of any one of claims 1-9, wherein the contacting is for at least 2 hours.

11. The method of any one of claims 1-10, wherein the monocyte is pro-inflammatory prior to being contacted with the isolated MSC exosome, and is regulatory after being contacted with the isolated MSC exosome.

12. A method of treating a fibrotic disease, the method comprising administering to a subject in need thereof an effective amount of a monocyte, wherein the monocyte is treated with an isolated mesenchymal stem cell (MSC) exosome prior to being administered.

13. A method of treating an autoimmune disease, the method comprising administering to a subject in need thereof an effective amount of a monocyte, wherein the monocyte is treated with an isolated mesenchymal stem cell (MSC) exosome prior to being administered.

14. The method of claim 12 or claim 13, further comprising isolating the monocyte prior to treating the monocyte with the MSC exosome.

15. The method of claim 14, wherein the monocyte is isolated from the subject.

16. The method of claim 15, wherein the monocyte is isolated from the bone marrow of the subject.

17. The method of any one of claims 12-16, wherein the monocyte is treated with the MSC exosome for at least 2 hours prior to being administered to the subject.

18. The method of any one of claims 12-17, wherein the monocyte is administered systemically.

19. The method of claim 18, wherein the monocyte is administered via intravenous infusion.

20. The method of any one of claims 12-18, wherein the monocyte is administered intratracheally or intranasally.

21. The method of any one of claims 12-20, wherein the monocyte is administered once to the subject.

22. The method of any one of claims 12-21, wherein the monocyte is administered multiple times to the subject.

23. The method of any one of claims 12-22, wherein the method further comprises administering to the subject an effective amount of a second agent.

24. The method of claim 23, wherein the second agent is an isolated MCS exosome.

25. The method of claim 23, wherein the second agent is nintedanib, Pirfenidone, an anti-fibrotic agent, an immunosuppressant, and/or an anti-inflammatory agent.

26. The method of any one of claims 12 and 14-25, wherein the fibrotic disease is selected from the group consisting of: systemic sclerosis; liver fibrosis, heart fibrosis, kidney fibrosis, and myelofibrosis.

27. The method of claim 26, wherein the fibrotic disease is pulmonary fibrosis.

28. The method of claim 27, wherein the pulmonary fibrosis is idiopathic pulmonary fibrosis (IPF).

29. The method of any one of claims 12 and 12-28, wherein the monocyte reduces inflammation associated with the fibrotic disease.

30. The method of any one of claims 12 and 12-29, wherein the monocyte reduces apoptosis associated with the fibrotic disease.

31. The method of any one of claims 12-30, wherein the subject is a mammal.

32. The method of claim 31, wherein the subject is a human subject.

33. The method of claim 32, wherein the human is a neonate, an infant, or an adult.

34. The method of claim 32, wherein the human subject is less than four weeks of age.

35. The method of claim 32, wherein the human subject is four weeks to 3 years of age.

36. The method of claim 32, wherein the human subject is 3-18 years of age.

37. The method of claim 32, wherein the human subject is an adult.

38. The method of any one of claims 32-37, wherein the human subject is born prematurely.

39. The method of claim 38, wherein the human subject was born before 37 weeks of gestation.

40. The method of claim 38, wherein the human subject was born before 26 weeks of gestation.

41. The method of claim 31, wherein the subject is a rodent.

42. The method of claim 41, wherein the rodent is a mouse or a rat.

43. The method of any one of claims 12-42, wherein the monocyte is pro-inflammatory prior to being treated with the isolated MSC exosome, and is regulatory after being treated with the isolated MSC exosome.

44. A monocyte treated with an isolated mesenchymal stem cell (MSC) exosome.

45. The monocyte of claim 44, wherein the monocyte is from bone marrow.

46. The monocyte of claim 44 or claim 45, wherein the isolated MSC exosome is isolated from MSC-conditioned media.

47. The monocyte of any one of claims 44-46, wherein the MSC is from Wharton's Jelly or bone marrow or adipose tissue.

48. The monocyte of any one of claims 44-47, wherein the monocyte is pro-inflammatory prior to being treated with the isolated MSC exosome, and is regulatory after being treated with the isolated MSC exosome.

49. A composition comprising the monocyte of any one of claims 42-48.

50. The composition of claim 49, further comprising a second agent.

51. The composition of claim 49 or claim 50, wherein the composition is a pharmaceutical composition.

52. The composition of any one of claims 49-51, wherein the composition further comprises a pharmaceutically acceptable carrier.

53. Use of the monocyte of any one of claims 44-48 or the composition of any one of claims 49-52 for treating a fibrotic disease.

54. The monocyte of any one of claims 44-48 or the composition of any one of claims 49-52, for use in the manufacturing of a medicament for treating a fibrotic disease.

55. Use of the monocyte of any one of claims 44-48 or the composition of any one of claims 49-52 for treating an autoimmune disease.

56. The monocyte of any one of claims 44-48 or the composition of any one of claims 49-52, for use in the manufacturing of a medicament for treating an autoimmune disease.
